(19) United States
(12) Patent Application Publication
(10) Pub. No.: US 2022/0025053 A1
(43) Pub. Date: Jan. 27, 2022

(54) Methods of Inhibiting Metastasis in Cancer

(71) Applicant: Wisconsin Alumni Research Foundation, Madison, WI (US)

(72) Inventors: Pamela Kay Kreeger, Middleton, WI (US); Molly Jane Carroll, Madison, WI (US); Kaitlin C. Fogg, Fitchburg, WI (US)

(21) Appl. No.: 17/495,893

(22) Filed: Oct. 7, 2021

Related U.S. Application Data
(62) Division of application No. 16/256,065, filed on Jan. 24, 2019, now abandoned.
(60) Provisional application No. 62/621,769, filed on Jan. 25, 2018.

(51) Int. Cl. C07K 16/28; A61P 35/04; C07K 16/24; C12N 15/113; A61K 31/439

(52) U.S. Cl. CPC C07K 16/2854; A61P 35/04; C07K 16/24; C12N 15/1138; A61K 2039/505; C07K 16/2866; C12N 2310/14; C07K 2317/76; A61K 31/439

(57) ABSTRACT

As described herein, a method of inhibiting metastasis in cancer includes administering to a human subject diagnosed with a cancer of an organ of the peritoneal cavity a therapeutically effective amount of an inhibitor of CCR5 or P-selectin. Preferably the subject has a tumor positive for a ligand of P-selectin such as a CD24+ or PSGL-1+ tumor. Analysis of samples from HGSOC patients confirmed increased MIP-1β and P-selectin, suggesting that this novel multi-cellular mechanism can be targeted to slow or stop metastasis in cancers such as high-grade serous ovarian cancer, for example by using anti-CCR5 and P-selectin therapies developed for other indications.

### CROSS-REFERENCE TO RELATED APPLICATIONS

This application is a divisional of U.S. application Ser. No. 16/256,065, filed on Jan. 24, 2019, which claims priority to U.S. Provisional Application 62/621,769 filed on Jan. 25, 2018, which is incorporated herein by reference in its entirety.

### STATEMENT REGARDING FEDERALLY SPONSORED RESEARCH & DEVELOPMENT

This invention was made with government support under CA195766 awarded by the National Institutes of Health. The government has certain rights in the invention.

### FIELD OF THE DISCLOSURE

The present disclosure is related to methods of inhibiting metastasis in cancer, particularly of cancers of the peritoneal cavity such as high-grade serous ovarian cancer.

### BACKGROUND

High grade serous ovarian cancer (HGSOC) is the most lethal gynecological cancer worldwide, with an overall 5-year survival of 46%. This dismal prognosis is a result of failure to diagnose most patients prior to the onset of metastasis throughout the peritoneum. Current therapeutics for HGSOC are primarily limited to platinum-based therapies that target proliferating cells. However, these therapies are ineffective at inhibiting adhesion and invasion in in vitro models of metastasis and no therapies exist to specifically target metastasis. Instead, to combat the spread of HGSOC throughout the peritoneum, a debulking surgery is performed either before chemotherapy or after neoadjuvant treatment. Surgical outcome is a strong predictor of prognosis; however, because many tumors have disseminated widely prior to surgery, complete surgical resection is not always possible. Additionally, even with aggressive surgery, microscopic disease remains for most patients, leading to recurrence and complications such as bowel obstructions that can be fatal during a new period of metastasis. Thus, identifying the mechanisms by which HGSOC populates the peritoneum may lead to new therapies and improved outcomes.

### BRIEF SUMMARY

In one aspect, a method of inhibiting metastasis in cancer comprises administering to a human subject diagnosed with a cancer of an organ of the peritoneal cavity a therapeutically effective amount of an inhibitor of CCR5 or P-selectin, wherein the subject has a tumor positive for a ligand of P-selectin such as a CD24+ or PSGL-1+ tumor.

### BRIEF DESCRIPTION OF THE DRAWINGS

FIG. 1 is a schematic of the pathway to metastasis involving CCR5 and P-selection identified by the inventors of the present application.

FIGS. 2A and B are an overview of co-culture device manufacturing and an in vitro model. FIG. 2A shows a process to cast and cure polydimethylsiloxane (PDMS) in ring-shaped molds in order to produce the PDMS ring component of the co-culture device. Dimensions indicated are outer dimensions (inner opening is 9 mm×11 mm). The ring height is 250 μm and the pillars provide a stop to maintain the coverslip in place during transport of cultures. FIG. 2B shows the metastatic adhesion model constructed by seeding LP-9 mesothelial cells to confluency within the collagen I coated center of the PDMS ring in a 24 well plate and then co-culturing LP-9 with primary alternatively-activated macrophages (AAMs) for 24 hours via placement of the AAM-seeded coverslip on top of the PDMS ring. Fluorescently labeled ovarian cancer cell lines (CaOV3, OV90, OVCAR5) were then added and allowed to adhere for three hours. Non-adherent cells were removed by washing with phosphate-buffered saline (PBS), and adherent cells were visualized using immunofluorescent microscopy. Similar methods can be used for other cell types such as colon cancer cells.

FIGS. 3A-I show AAMs increase HGSOC adhesion to LP-9 through upregulation of mesothelial P-selectin. FIG. 3A shows confocal reconstruction of CaOV3 (top) adhered to LP-9 (bottom). Scale bar=20 μm. FIG. 3 B and C show representative image (B) of OVCAR5 adhered to LP-9 and quantification (C). Scale bar=100 μm, n=3 replicates, one AAM donor. FIG. 3I) shows potential direct/indirect effects of AAMs. FIG. 3E shows AAMs were included during three-hour adhesion (direct) or during 24-hour time prior to tumor cell addition (indirect), n=3 replicates, one AAM donor. FIG. 3F shows a screen of 86 ECM/adhesion genes from LP-9 cultured in the absence or presence of AAMs for 24 hours. FIG. 3G shows validation of increased SELP in LP-9, n=3 unique AAM donors. FIG. 3H shows adhesion of HGSOC to adsorbed isotype or P-selectin Fc chimera, n=4. FIG. 3I shows LP-9 in the absence or presence of AAMs that were treated with isotype or P-selectin blocking antibody prior to addition of HGSOC. Data is average±SD, *p<0.05 vs. −AAMs (C,E,G), isotype (H), or −AAMs/isotype (I), \hat{ }p<0.05 vs. +AAMs/isotype (I) by two-sided t-test (C,G,H), two-sided t-test with Bonferroni correction (E,I).

FIGS. 4A-F show partial least squares regression (PLSR) prediction and experimental validation of role for MIP-1β in increased HGSOC adhesion. FIG. 4A shows ligands (z-score normalized) detected in the absence or presence of AAMs. Data is an average of n=3 replicates per donor; each column represents a unique donor/cell line combination. FIG. 4B is a comparison of PLSR-predicted to experimentally-observed HGSOC adhesion to LP-9. FIG. 4C shows correlations of ligands and observed adhesion (% Adhered) with principal component PC1 and PC2 from the PLSR model. FIG. 4D shows VIP>1 (variable importance in projection, dark grey) indicating important variables to predict adhesion. Those that positively correlated with HGSOC adhesion are shown in bold (and bolded in the heatmap (A) and labeled in (C)). FIG. 4E shows OV90 co-cultures were treated with neutralizing antibodies against IL-13 (IL13), PDGF-BB (PDGF), MIP-1β (MIP), or isotype (Iso) during co-culture, n=3 replicates, one AAM donor. FIG. 4F shows HGSOC adhesion to LP-9 treated with vehicle or 100 ng/mL MIP-1β, n=3. Data is average±SD, *p<0.05 vs. −AAMs of same isotype/antibody (E) or vehicle (F), \hat{ }p<0.05 vs. +AAMs/isotype (E) by two-sided t-test (F) with Bonferroni correction (E).

FIGS. 5A-E show PLSR model overview and validation in multiple HGSOC lines. FIG. 5A shows the accuracy (R²Y) and predictability (Q²Y) of two-component partial least squares regression (PLSR) model for ovarian cancer adhesion to LP-9. FIG. 5B shows the scores plot showing correlation of observations (adhesion of CaOV3, OV90, OVCAR5±AAMs) along principal component 1 (PC1) and PC2 in PLSR model. Legend is same as in FIG. 4B. FIG. 5C shows the effect of neutralizing antibodies (IL-13, PDGF-BB, MIP-1β) or isotype on CaOV3 (left) and OVCAR5 (right) adhesion to LP-9. Antibody doses are the same as in FIG. 2E, n=3 replicates from one AAM donor. FIG. 5D shows conditioned media was collected from micro-devices with AAMs alone or LP-9, AAMs, and ovarian cancer cells (CaOV3, OV90, or OVCAR5) and assayed for MIP-1β by ELISA. Results indicate that AAMs generate MIP-1β and that much of this MIP-1β is consumed by the other cells in the device, n=3 replicates from one AAM donor. MIP-1β was not detectable in monocultures of CaOV3, OV90, OVCAR5, or LP-9. FIG. 5E shows LP-3, a second mesothelial cell line, was treated with vehicle or 100 ng/mL MIP-1β for 24 hours, and adhesion of the CaOV3 ovarian cancer cell line was assayed after three hours, n=3. Data is average±SD, *p<0.05 vs. −AAMs/isotype (C), AAMs alone (D), vehicle (E), \hat{ }p<0.05 vs. +AAMs/isotype (C) by two-sided t-test (E) with Bonferroni correction (C,D)

FIGS. 6A-J show that MIP-1β signals through CCR5/PI3K to up-regulate P-selectin. FIGS. 6A and B shows quantification of P-selectin in LP-9 in response to MIP-1β neutralizing antibody or isotype by qRT-PCR (A) and immunofluorescence (B), n=3 replicates, one AAM donor. FIG. 6C shows flow cytometry analysis of P-selectin in LP-9 treated with vehicle or 100 ng/mL MIP-1β (MIP-1β). FIG. 6D shows SELP expression in LP-9 after 24 hours of treatment with increasing MIP-1β, n=3. FIG. 6E shows treatment of LP-9 with 100 ng/mL MIP-1β and the P-selectin small molecular inhibitor KF38789 demonstrated P-selectin was necessary for MIP-1β increased adhesion, n=3. FIG. 6F shows treatment of LP-9 with 100 ng/mL MIP-1β and a CCR5 blocking antibody or isotype demonstrated that CCR5 was necessary for increased SELP, n=3. FIG. 6G shows treatment of LP-9 with 100 ng/mL MIP-1β and maraviroc or DMSO demonstrated that maraviroc negated SELP upregulation, n=3. FIG. 6H shows immunofluorescence of p65 in LP-9 treated with vehicle or 100 ng/mL MIP-1β (MIP-1β) over time. Scale bar=50 μm. FIG. 6I shows LP-9 were treated with vehicle or 100 ng/mL MIP-1β in combination with DMSO control, 10 μM PI3K (LY) or MEK (PD) inhibitors and SELP expression analyzed by qRT-PCR FIG. 6J shows ERK and AKT phosphorylation (Thr308, Ser473) of LP-9 treated with vehicle or 100 ng/mL MIP-1β. Data is average±SD, *p<0.05 vs. −AAMs/isotype (A,B), vehicle (D,J), vehicle/isotype (F), or vehicle/DMSO (E,G,I), \hat{ }p<0.05 vs. +AAMS/isotype (A,B), 10 ng/mL MIP-1β (D), MIP-1β/isotype (F) or MIP-1β/DMSO (E,G,I) by two-sided t-test with Bonferroni correction (A,B,D-G,I) or two-sided t-test at each time (J).

FIG. 7A-C shows MIP-1β upregulation of SELP in multiple mesothelial cell lines. FIG. 7A is representative images of LP-9 cultured in the absence and presence of AAMs and treated with 1 μg/mL isotype control or MIP-1β neutralizing antibody for 24 hours. Note that the antibody and staining protocol are specific for all surface P-selectin, rather than intracellular pools. Scale bar=100 μm. In FIG. 7B, LP-3 were treated with vehicle or 100 ng/mL MIP-1β for 24 hours, and SELP expression was analyzed using qRT-PCR, n=3, normalized to GAPDH. Data is average±SD, *p<0.05 vs. vehicle by two-sided t-test. FIG. 7C shows flow cytometry demonstrating that vehicle-treated LP-9 P-selectin (CD62p) levels are comparable to isotype control and that the extent of MIP-1β induced P-selectin in mesothelial cells is comparable to IL-4 treated HUVECs (1).

FIGS. 8A-F show HGSOC cells adhere to P-selectin through CD24. FIG. 8A shows flow cytometry analysis of CD162 and CD24 in HGSOC cells vs. isotype controls. FIG. 8B shows representative immunofluorescent staining for CD15s in HGSOC cells. Scale bar=50 μm. FIG. 8C shows the correlation of median CD24 fluorescence from flow cytometry (A) to fold-change in adhesion to LP-9 co-cultured with AAMs. FIG. 8D shows OV90 were treated with siCD24 or siC siRNA and their adhesion to LP-9 treated with vehicle or 100 ng/mL MIP-1β was assayed; n=3. Data is average±SD, *p<0.05 vs. Veh/siC (D), \hat{ }p<0.05 vs. MIP-1β/siC by two-sided t-test with Bonferroni correction

FIG. 8E shows CaOV3 were pumped at 0.125 dyn/cm² into parallel flow channels containing LP-9 treated with vehicle or MIP-1β. Channels coated with BSA served as a negative control. Cells had a significantly lower velocity distribution on MIP-1β treated LP-9 compared to vehicle, n=200 cells/condition, p<0.001 by Kolmogorov-Smimov test. FIG. 8F shows rolling flux of CaOV3 for the conditions in (E), n=200 cells/condition. Data is average±SD, vehicle and BSA (F), \hat{ }p<0.05 by two-sided t-test.

FIG. 9A shows flow cytometry analysis of CD24 in additional HGSOC cells vs. isotype controls. FIG. 9B shows the impact of MIP-1β on adhesion of additional HGSOC cell lines, n=3. Data is average±SD *p<0.05 compared to vehicle (B).

FIGS. 10A and B show CD24 expression in HGSOC lines. FIG. 10A shows representative immunofluorescent images of CD24 staining in CaOV3, OV90, and OVCAR5 ovarian cancer cell lines. Scale bar=100 μm. FIG. 10B shows representative immunofluorescent images of secondary-only staining (AlexaFluor® 488) in CaOV3, OV90, and OVCAR5 ovarian cancer cell lines. Scale bar=100 μm.

FIG. 11 shows spheroid flow after treatment with MIP-1β at 25 μL/min (0.0317 dyn/cm²), 700 spheroids/mL, 50 cells/spheroid.

FIG. 12 shows spheroid flow after treatment with MIP-1β at 50 μL/min (0.0634 dyn/cm²), 700 spheroids/mL, 50 cells/spheroid.

FIGS. 13A-C show MIP-1β increases P-selectin in vivo and adhesion in vivo and ex vivo. FIG. 13A shows mice were i.p. injected with vehicle control or 1 μg and assayed for SELP 24 hours later by qRT-PCR. FIG. 13B shows immunohistochemistry for P-selectin was performed on the peritoneal wall, omentum, and mesentery tissues of the mice described in (A), scale bar=100 μm. FIG. 13C shows ex vivo adhesion of CaOV3 to peritoneal wall biopsies from mice inoculated with vehicle control or 1 μg MIP-1β. Scale bar=1 mm. Images (left) and quantified adhesion (right) from n=3 mice from each treatment condition. Data is average±SD, * p<0.05 vs. vehicle by a two-sided t-test.

FIG. 14A-D shows MIP-1β increases P-selectin in vivo and adhesion in vivo and ex vivo. FIG. 14A, IHC for P-selectin was performed on the peritoneal wall, omentum, and mesentery of mice that were intraperitoneally injected with vehicle or 1 μg MIP-1β. Scale bar, 100 μm. FIG. 14B, Ex vivo adhesion of CaOV3 to peritoneal wall biopsies from mice treated as in A. Scale bar, 1 mm. Images (left) and quantified adhesion (right) from n=3 mice. FIGS. 14C and 14D, In vivo adhesion of ID8 to the peritoneal wall, omentum (shown in C), and mesentery was assayed after 90 minutes in mice intraperitoneally injected with vehicle control or 1 μg MIP-1β, followed by DMSO control or KF38789 (1 mg/kg, MIP-1β/KF38789). Scale bar, 0.5 cm. Data are Average+/−SD; *, P<0.05 vs. vehicle (B) or vehicle/DMSO (D); \hat{ }, P<0.05 vs. MIP-1β/DMSO by a two-sided t test (B) with Bonferroni correction (D).

FIG. 15 shows MIP-1β in vivo impacts on P-selectin expression. FIG. 15 shows no primary controls in immunohistochemistry sections of mice inoculated with vehicle control (Veh) or 1 μg MIP-1β. Scale bar=100 μm.

FIGS. 16A-E show HGSOC patients have elevated MIP-1β and P-selectin. FIG. 16A shows HGSOC ascites had elevated MIP-1β concentrations compared to benign conditions. n=4 benign, n=20 HGSOC. FIG. 16B shows LP-9 were treated with 10% (v/v) of PBS (Veh) or ascites in SFM in conjunction with an isotype control or MIP-1β blocking antibody (A) for 24 hours, n=3 for each patient sample with OV90 cell line. FIG. 16C shows the Kaplan-Meier plotter tool was utilized with Gene Omnibus and The Cancer Genome Atlas databases to calculate PFS for HGSOC patients with low and high expression of CD24. FIG. 16D shows representative omental tissue sections from patients with non-HGSOC or HGSOC conditions stained for P-selectin (P-sel) and calretinin (Cal, mesothelial cells). Scale bar=25 μm. FIG. 16E shows quantification of P-selectin levels in the mesothelium (calretinin-positive) normalized to mesothelium area, n=3 patients/category. Data is average±SD, * p<0.05 vs. benign patients (A), vehicle/isotype (B), non-HGSOC samples (E), \hat{ }p<0.05 vs. corresponding patient ascites/isotype (B) by two-sided t-test (A,E) with Bonferroni correction (B), or log-rank test (C).

FIG. 17 shows HGSOC ascites increases HGSOC adhesion. Impact of HGSOC ascites (10% v/v) in conjunction with MIP-1β blocking antibody on CaOV3 (left) and OVCAR5 (right) adhesion to LP-9, n=3. Data is average±SD *p<0.05 compared to Veh/Isotype, \hat{ }p<0.05 vs corresponding patient ascites/MIP-1β Ab by two-sided t-test with Bonferroni correction.

FIG. 18 shows copy number alterations in peritoneal metastasized cancers. Chromosomal copy numbers of ovarian, endometrial, colorectal, and pancreatic cancers which can metastasize to the peritoneal cavity and omentum.

FIGS. 19A-C show immunofluorescence of calretinin and P-selectin in non-HGSOC and HGSOC omental biopsies. FIG. 19A shows primary controls demonstrating low non-specific binding of secondary antibodies for P-selectin (AF 488) and calretinin (AF 647). Scale bar=100 μm. FIG. 19B shows in some sections, P-selectin positive staining was observed in regions separated from the mesothelial layer. To confirm that this signal resulted from P-selectin on anuclear platelets, sections were labeled for P-selectin and CD31 (endothelial cells). As suspected, this P-selectin signal was restricted within blood vessel walls in DAPI-negative regions, consistent with the interpretation that the signal separate from mesothelial cells was due to anuclear platelets. Staining was conducted as in Methods, using anti-CD31 (ab28364, Abcam, at 1:50). Scale bar=100 μm. FIG. 19C shows images for additional omental samples from non-HGSOC and HGSOC patients. Quantification of P-selectin levels in the mesothelial layer for these patients is included in FIG. 5E. Scale bar=25 μm.

FIG. 20 shows the impact of MIP-1β induced P-selectin on colorectal cancer cell adhesion. Bar plot indicates average±standard deviation, n=3 wells per condition, * p<0.05 compared to Veh/DMSO, \hat{ }p<0.05 compared to MIP-1β/DMSO.

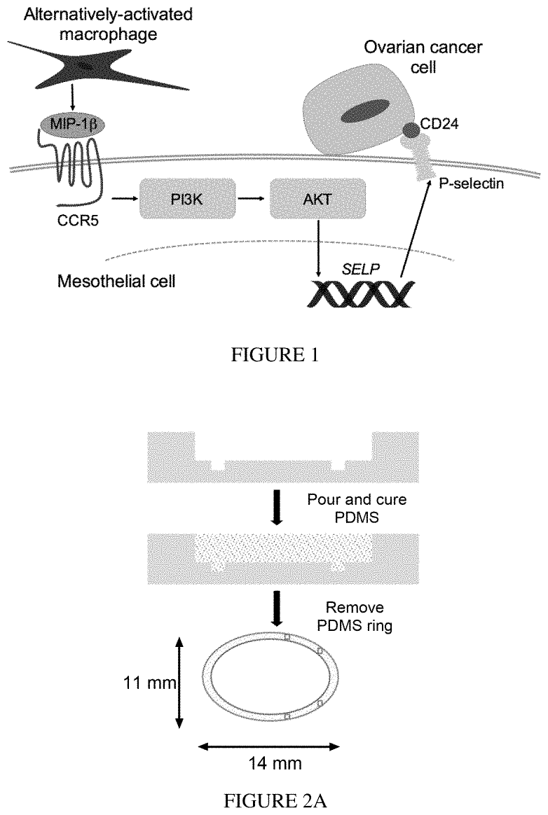

The above-described and other features will be appreciated and understood by those skilled in the art from the following detailed description, drawings, and appended claims.

### DETAILED DESCRIPTION

The inventors have unexpectedly found that inhibitors of P-selectin and CCR5 can be used to inhibit metastasis of a P-selectin ligand positive, e.g., CD24+, cancer of an organ of the peritoneal cavity, specifically high-grade serous ovarian cancer. Specifically, the inventors found that AAM-secreted MIP-1β activates CCR5/PI3K signaling in mesothelial cells, resulting in expression of P-selectin on the mesothelial cell surface. Tumor cells attached to this de novo P-selectin through CD24, for example, resulting in increased tumor cell adhesion in static conditions and rolling under flow. Immunohistochemical and qRT-PCR analysis showed that C57/BL6 mice treated with MIP-1β increased P-selectin expression in peritoneal tissues, which enhanced CaOV3 adhesion ex vivo and ID8 adhesion in vivo. Analysis of samples from HGSOC patients confirmed increased MIP-1β and P-selectin, suggesting that this novel multi-cellular mechanism could be targeted to slow or stop metastasis in HGSOC by repurposing anti-CCR5 and P-selectin therapies developed for other indications. Similar results were obtained with colon cancer cells lines suggesting that the methods described herein are not limited to ovarian cancer.

HGSOC primarily metastasizes via the transcoelomic route, whereby tumor cells detach from the primary tumor, float through the ascites, and adhere to mesothelial-lined surfaces in the peritoneal cavity. During this process, HGSOC cells are likely influenced by numerous elements of the microenvironment, including alternatively activated macrophages (AAMs). In contrast to pro-inflammatory classically activated macrophages (CAMs), AAMs possess a pro-tumor, anti-inflammatory phenotype and have been linked to remodeling behaviors in vivo such as wound healing and tumor progression. It has been found that AAMs are present in the ascites of many HGSOC patients, and experimental evidence supports a role for macrophages in HGSOC metastasis. In vivo analysis of ovarian cancer xenograft models treated with clodronate to reduce macrophage levels showed decreased metastasis. Clinical studies have found that an increase in tumor AAM-density correlates with advanced disease staging and poor prognosis. While in vitro co-culture of breast cancer cells with AAMs resulted in increased epithelial-mesenchymal transition and it has previously been shown that AAM co-culture with HGSOC cells can induce proliferation, the mechanisms by which AAMs in the microenvironment may promote HGSOC metastasis are unknown.

The inventors hypothesized that paracrine signaling from AAMs enhances HGSOC adhesion to mesothelial cells. Through the use of an in vitro model mimicking the HGSOC metastatic microenvironment and multivariate analysis, the inventors determined that AAM-secreted MIP-1β increased adherence of HGSOC to mesothelial cells. Further, through multiple experimental approaches, the mechanism by which MIP-1β's actions were achieved was decoded. Furthermore, this mechanism was validated using in vivo models and HGSOC patient data. These results indicate a mechanism by which AAMs educate the microenvironment to enhance HGSOC progression and identified multiple targets for which therapies to slow or stop HGSOC metastasis can be developed and tested.

In contrast to co-culture models where the cells can have physical contact, the co-culture described herein maintains cells on separate surfaces that can be brought together or separated as needed to examine the dynamics of paracrine cellular interactions. Through this feature, the inventors were able to determine that the effects of AAM secreted factors on mesothelial cells were essential for enhanced tumor cell adhesion. Identifying the specific secreted factor or factors responsible for this enhanced adhesion was non-trivial, as the co-cultures were positive for 27 of the 36 ligands assayed. Without being held to theory, the inventors hypothesized that levels of factors present in the cultures would correlate with the percentage of adherent HGSOC cells. Therefore, PLSR, a multivariate modeling approach that emphasizes co-variation between an independent and dependent data set, was utilized. While PLSR has been most widely used in the systems biology field to examine the relationship between signaling events and downstream cellular phenotypes, it has also been used to examine how protein levels correlate to outcomes such as drug treatment in HGSOC. The use of this modeling technique unexpectedly suggested a strong correlation for only four secreted factors, simplifying attempts to unravel the mechanism of action.

Because correlation does not equal causation, the inventors sought to validate the potential role of the suggested ligands through a combination of neutralization experiments and treatment with the candidate factor in the absence of other AAM secreted factors. These results indicated that MIP-1β was necessary and sufficient for the observed increase in tumor adhesion. Interestingly, an analysis of the current literature did not indicate a clear role for MIP-1β in HGSOC. For example, the concentration of MIP-1β in serum was reported to be lower in HGSOC patients compared to a benign patient. However, matched ascites and serum samples from HGSOC patients indicated that the concentration of MIP-1β was higher in ascites compared to serum levels, suggesting that MIP-1β is concentrated in the peritoneal microenvironment of HGSOC patients. Therefore, the inventors sought to compare the levels of peritoneal MIP-1β between benign conditions and HGSOC patients and determined the MIP-1β was significantly elevated in HGSOC. MIP-1β has been detected in HGSOC biopsies, however, detectable levels were not observed in media from HGSOC or mesothelial cells, suggesting that other cells in the microenvironment may be the source of MIP-1β. A prior investigation found that the presence of CD68+ macrophages in the stroma did not correlate to MIP-1β levels; however, this study did not further characterize the macrophages into the classically-activated macrophage (CAM) or AAM phenotype and the ratio of AAMs:CAMs varies between HGSOC patients. In the analysis of factors secreted in the in vitro model described herein, the inventors showed that AAMs secreted MIP-1β.

The inventors next sought to understand what elements of the mesothelial cells were altered by MIP-1β to increase adhesion. Through a qRT-PCR screen, changes in SELP expression were identified and it was validated that P-selectin was responsible for HGSOC cell adhesion. Normal omentum, in contrast, expresses very low levels of P-selectin. Immunofluorescent staining of omental biopsies from HGSOC patients and peritoneal organs from MIP-1β treated mice demonstrated increased P-selectin expression. Furthermore, in vivo treatment with MIP-1β increased the adhesion of CaOV3 cells ex vivo. Additionally, the inventors determined that CCR5/PI3K signaling was responsible for the upregulation of P-selectin by MIP-1β. This link that has not been documented in any cell type but is supported by evidence that endothelial cell expression of P-selectin is controlled by PI3K/AKT pathway.

While the experimental results clearly demonstrated an important role for P-selectin in the increased adhesion of tumor cells to mesothelial cells, the extent of adhesion of tumor cells to adsorbed P-selectin was lower than expected. However, the initial experiments were done under static conditions and P-selectin is best known for inducing rolling under flow through the use of both slip- and catch-bond, as has been shown to occur in breast cancer cells flowing over endothelial cells to aid in hematogenic metastasis. As HGSOC metastasizes by the transcoelomic route, tumor cells will be subject to the flow conditions that are inherent to the peritoneal cavity. When HGSOC adhesion to mesothelial cells was examined under flow, no evidence of rolling was observed in the absence of MIP-1β, when mesothelial cells are P-selectin negative. In contrast, an increased number of tumor cells and tumor cell aggregates rolled and had lower velocities on MIP-1β treated mesothelial cells that express P-selectin. This suggests that P-selectin may play an even larger role in HGSOC metastasis in vivo than the initial experiments in static conditions suggested.

To complete the inventors' understanding of this multi-cellular mechanism, they sought to identify the tumor cell ligand responsible for the adhesion to mesothelial cells. While PSGL-1 has been shown to be expressed in neutrophils and binds to P-selectin on endothelial cell, the data demonstrated that the panel of HGSOC lines did not express PSGL-1. However, all three lines did express CD24, which has been shown to initiate breast cancer rolling along P-selectin on endothelial cells. Interestingly, clinical studies have identified CD24 as a biomarker of poor prognosis and indicative of an invasive phenotype in ovarian cancer. Additionally, an analysis of TCGA data found that high expression of CD24 correlated with worse progression-free survival. Other cancers, such as endometrial, colorectal, and pancreatic can metastasize to the omentum and peritoneal cavity, and analysis of the Cancer Cell Line Encyclopedia found they have varying copy levels of CD24. Investigation of HGSOC cell lines showed that CaOV3 and OVCAR8, previously found to metastasize to the peritoneal wall and omentum in in vivo studies, had higher copy number of CD24 within HGSOC lines, and that copy number correlated with mRNA expression in the three HGSOC cell lines used in this study. Despite these correlations, the mechanisms by which CD24 influence HGSOC metastasis are not well understood, and much of the effect of CD24 has been attributed to its identification as a marker of cancer stem cells. Using quantitative approaches, the inventors showed that CD24 levels correlated to the fold-change in adhesion in the presence of AAMs, and further that knockdown of CD24 inhibited AAM-enhanced adhesion to mesothelial cells. Thus, the study illustrates a novel mechanism by which CD24 enhances the metastasis of HGSOC via its interaction with P-selectin on mesothelial cells in the tumor microenvironment.

In an embodiment, a method of inhibiting metastasis in cancer comprises administering to a human subject diagnosed with a cancer of an organ of the peritoneal cavity a therapeutically effective amount of an inhibitor of CCR5 or P-selectin, wherein the subject has a tumor positive for a ligand of P-selectin. Exemplary ligands of P-selectin include CD24 and PGSL-1.

Exemplary organs of the peritoneal cavity include the ovaries, uterus, endometrium, cervix, small intestine, colon, anus, rectum, liver, gallbladder, pancreas, kidneys, or bladder.

In a specific embodiment, the subject is suffering from high-grade serous ovarian cancer. In a more specific embodiment, the subject has a stage III or stage IV cancer.

In an embodiment, the P-selectin inhibitor is a monoclonal antibody such as crizanlizumab or inclacumab. These antibodies against P-selectin have been developed to treat sickle cell anemia and myocardial damage following a heart attack, respectively. Both antibodies were well tolerated in patients when administered systemically. The analysis of patient samples confirmed the P-selectin is dysregulated (i.e., present) in patients with HGSOC. In vitro tests with an anti-P-selectin antibody and a small molecule inhibitor demonstrated that this approach was able to inhibit tumor cell adhesion; the antibody used has a similar mechanism of action as crizanlizumab and inclacumab, suggesting that the in vitro results may be mirrored in vivo with these humanized monoclonal antibodies. Excitingly, crizanlizumab may have additional potency as it has been shown to not only block ligand binding but also disrupt existing P-selectin-ligand interactions.

In another embodiment, the P-selectin inhibitor is a small molecule such as rivipansel or tinzaparin.

Alternatively, drugs targeting CCR5, such as the allosteric inhibitor maraviroc, have been developed due to the role of CCR5 as a co-receptor for HIV. The inventors' analysis of patient samples demonstrated that MIP-1β is elevated in HGSOC and maraviroc inhibited the upregulation of SELP in mesothelial cells in an in vitro model. Thus, in an embodiment, the CCR5 inhibitor is maraviroc, vicriviroc, or aplaviroc.

The inhibitor of CCR5 or P-selectin can be administered in the form of a pharmaceutical composition. As used herein, “pharmaceutical composition” means therapeutically effective amounts of the inhibitor with a pharmaceutically acceptable excipient, such as diluents, preservatives, solubilizers, emulsifiers, and adjuvants. As used herein “pharmaceutically acceptable excipients” are well known to those skilled in the art.

Pharmaceutical compositions include reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

In one embodiment, the pharmaceutically acceptable excipient is suitable for parenteral administration. Alternatively, the pharmaceutically acceptable excipient can be suitable for subcutaneous, intravenous, intraperitoneal, intramuscular, or sublingual administration. Pharmaceutically acceptable excipients include sterile aqueous solutions or dispersions and sterile powders for the extemporaneous preparation of sterile injectable solutions or dispersions. The use of such media and agents for pharmaceutically active substances is well known in the art.

Parenteral pharmaceutical compositions are typically sterile and stable under the conditions of manufacture and storage. The pharmaceutical composition may be in lyophilized form. The composition can be formulated as a solution, microemulsion, liposome, or other ordered structure suitable to high drug concentration. The excipient can be a solvent or dispersion medium containing, for example, water, ethanol, polyol (for example, glycerol, propylene glycol, and liquid polyethylene glycol), and mixtures thereof. A stabilizer can be included in the pharmaceutical composition.

Pharmaceutical compositions can include isotonic agents, for example, sugars, polyalcohols such as mannitol, sorbitol, or sodium chloride. Prolonged absorption of the injectable compositions can be brought about by including in the composition an agent which delays absorption, for example, monostearate salts and gelatin. The inhibitor can be formulated in a time release formulation, for example in a composition which includes a slow release polymer. The inhibitor can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

The inhibitor may be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the inhibitor can either be suspended or dissolved in the vehicle. Advantageously, adjuvants such as a local anaesthetic, preservative, and buffering agents can be dissolved in the vehicle. Subcutaneous administration can be daily administration.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

In an aspect, a pharmaceutical composition can further comprise a second active agent such as an anti-cancer agent.

Exemplary anti-cancer agents for co-administration with the inhibitor of CCR5 or P-selectin include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, olaparib, ormnaplatin, oxaliplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, rucaparib, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, velaparib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, zorubicin, other PARP inhibitors, and combinations comprising at least one of the foregoing. In an aspect, co-administration of an anti-cancer agent with the inhibitor of CCR5 or P-selectin provides for a reduction in the dosage of the anti-cancer agent.

In an aspect, the chemotherapeutic agent is carboplatin, cisplatin, oxaliplatin, paclitaxel, docetaxel, olaparib, rucaparib, veliparib, or a combination thereof.

As used herein, a “subject” includes mammals, specifically humans.

In an aspect, the subject has had tumor removal surgery, e.g., debulking, and/or neoadjuvant therapy, prior to administering.

In an aspect, the subject is a subject in need of palliative care, such as a patient with chemotherapy-resistant cancer. In an aspect, inhibiting metastasis may slow the incidence of complications such as bowel obstructions.

The invention is further illustrated by the following non-limiting examples.

### EXAMPLES

### Methods

Cell lines and reagents: Unless otherwise stated, all reagents were purchased from ThermoFisher (Waltham, Mass.). HGSOC cell lines CaOV3, OV-90, and OVCAR3 were purchased from American Type Culture Collection (ATCC; Rockville, Md.), OVCAR 4, OVCAR5 and OVCAR8 were obtained from NCI 60 panel (NIH). The LP-9 and LP-3 mesothelial cell lines were purchased from the Coriell Cell Repository (Camden, N.J.). All cell lines were authenticated by human short tandem repeat (STR) analysis at the Experimental Pathology Laboratory at the University of Wisconsin—Madison. Cells were maintained at 37° C. in a humidified 5% CO₂ atmosphere. CaOV3 and OVCAR5 were cultured in a 1:1 (v/v) ratio of MCDB105:Medium199 (Corning; Corning, N.Y.) supplemented with 1% penicillin/streptomycin and 10% heat-inactivated fetal bovine serum (FBS). OV-90, OVCAR3, OVCAR4, and OVCAR8 were cultured in a 1:1 (v/v) ratio of MCDB105:Medium199 supplemented with 1% penicillin/streptomycin and 15% heat-inactivated FBS. LP-9 and LP-3 lines were cultured in a 1:1 (v/v) ratio of Hams F12 (Corning):Medium199 with 1% penicillin/streptomycin, 15% FBS, 2 mM L-glutamine, 10 ng/mL epidermal growth factor (Peprotech; Rocky Hill, N.J.), and 0.4 μg/mL hydrocortisone (Corning).

Isolation and differentiation of AAMs from whole blood: Whole blood from healthy females over the age of 18 years was purchased from Innovative Research (Novi, Mich.). Monocytes were enriched by negative selection using the Rosette Sep® monocyte enrichment cocktail according to manufacturer's instructions (STEMCELL Technologies; Vancouver, Canada). To differentiate isolated monocytes into the AAM phenotype, monocytes were seeded on 9 mm square coverslips at a density of 200,000 cells/well for 6 days in AIM V® media supplemented with 1% penicillin-streptomycin and 20 ng/mL M-CSF (Peprotech). Macrophages were polarized for 48 hours with 2 ng/mL IL-4 and 2 ng/mL IL-13 (Peprotech). AAMs were washed with phosphate buffered saline (PBS) and changed to 1:1 Medium199:MCDB105 supplemented with 1% penicillin/streptomycin (serum free media, SFM) for 24 hours. Control conditions (−AAMs) were prepared by exposing cell-free coverslips to the differentiation protocol to account for the effects of non-specific adsorption of differentiation factors.

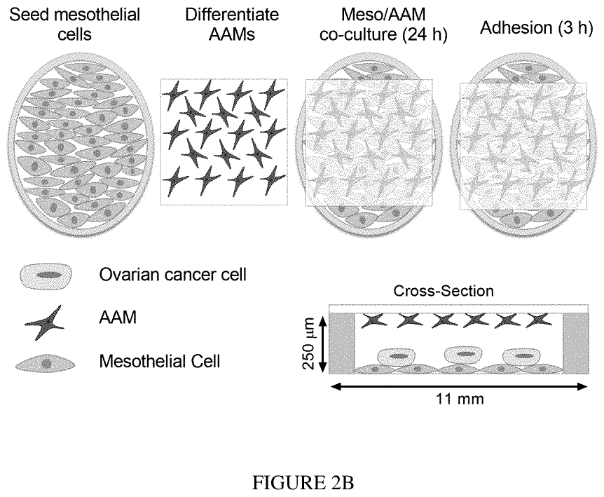

In vitro model of adhesion in transcoelomic metastasis: A co-culture device was modified to construct the in vitro model of metastatic adhesion (FIG. 2). The device includes a PDMS ring that is placed in a well of a 24 well tissue culture plate and a 9×9 mm coverslip placed on top. One or more cell types can be grown within the ring, while another population can be grown on the coverslip. Inverting the coverslip on top of the ring initiates co-culture between the populations. The tissue culture plastic within the PDMS ring was coated with 1 μg of PureCol® Collagen I (Advanced Biomatrix; San Diego, Calif.) for 2 hours at room temperature. LP-9 or LP-3 were seeded into the PDMS rings to confluency (93,500 cells/cm² in 40 μL). Twenty-four hours after seeding, cells were washed with SFM, AAM or control coverslips were placed on top of the PDMS ring, and 40 μL of fresh SFM was added. HGSOC cells were stained with 5 μM CellTracker™ Green CMFDA Dye, dissociated using TrypLE™ Select Enzyme, and seeded into devices at 10,000 cells/10 μL after 24 hours of mesothelial cells and AAMs co-culture. HGSOC cells were allowed to adhere for three hours, then coverslips were removed, and devices were washed twice with 2 mL PBS to remove non-adherent cells. Cells within the ring were fixed with 4% paraformaldehyde (Electron Microscopy Sciences; Hatfield, Pa.) for 15 minutes, washed twice with PBS, and fluorescent HGSOC cells were imaged on a Zeiss Axio Observer.Z1 inverted microscope with an AxioCam 506 mono camera, Plan-NEOFLUOR 20× 0.4-NA air objective, and Zen2 software (Zeiss; Oberkochen, Germany). Five images per well were captured, n=3 wells/condition. Percent adhesion was quantified by converting cells/image to total cells/area of the co-culture device and dividing by the number of HGSOC cells added to the device.

Confocal imaging of HGSOC adhesion: LP-9 and OV-90 were stained with 5 μM CellTracker™ Green and 1 μM CellTracker™ Deep Red, respectively. Following completion of the adhesion assay as described above, cultures were imaged with a Nikon AR1S confocal microscope and z-stack reconstructions were created in ImageJ (NIH).

In vivo and ex vivo mouse studies: Female C57BL/6 (6-12 weeks) were procured from the Research Animal Resource Center Breeding Services (UW-Madison). All animal protocols were approved by the Institutional Animal Care and Use Committee (IACUC) at the UW-Madison School of Medicine and Public Health. Mice were i.p. injected with 1 μg of recombinant mouse MIP-1β in 100 μL PBS or PBS control. After 24 hours, mice were euthanized by CO₂ asphyxiation, and the peritoneum, omentum, and mesentery were removed for qRT-PCR, immunohistochemistry, or ex vivo adhesion assays. Due to their small size, the omentum was analyzed by immunohistochemistry only and the mesentery by qRT-PCR and immunohistochemistry only. Peritoneal wall sections were cut using a biopsy punch, adhered into wells of an 8-well chamber slide, and covered with 400 μL serum-free media. Cell Tracker™ Deep Red (1 μM) stained CaOV3 were seeded into chamber slide wells (5,000 cells/100 μL) and allowed to adhere to the peritoneal tissue for 3 hours. Chamber slides were then washed twice with PBS and fixed with 10% formalin for 20 minutes. Fluorescent CaOV3 cells were counted and quantified as cells/cm² tissue.

For in vivo adhesion assays, female C57BL/6 mice will be i.p. injected with 1 μg recombinant mouse MIP-1β or PBS, and 24 hours later i.p. injected with 300,000 ID8 cells stained with 1 μM Cell Tracker™ Deep Red. After 90 minutes, mice will be euthanized and the peritoneal wall, omentum, and mesentery will be removed and whole-mounted onto slides. Fluorescently labeled ID8 cells on the excised tissues will be imaged on the Zeiss Axio Observer.Z1.

RNA extraction and qRT-PCR: RNA was collected and isolated using the Micro-RNeasy® Extraction kit (Qiagen; Valencia, Calif.), and cDNA was synthesized using the Qiagen® First Strand Kit according to manufacturer's instructions. cDNA was mixed with Qiagen® Mastermix and assayed using the Extracellular Matrix and Adhesion Molecules RT2 Profiler PCR Array (Qiagen) in a CFX real time PCR machine (Bio-Rad; Hercules, Calif.) for a total of 40 cycles, using Qiagen's Data Analysis Center for analysis. Data is expressed as fold change, with ±2-fold set as the threshold for significance. qRT-PCR was performed using human primers for SELP, CCR1, and CCR5, and GAPDH (all Qiagen), and mouse primers for Selp (Qiagen) and Aes (IDT; Coralville, Iowa), with SsoAdvanced™ Universal SYBR® Green Supermix (Bio-Rad). Three samples were run in duplicate from each condition.

Characterization of MMPs and cytokines: Conditioned media was collected from HGSOC adhesion assays using two unique AAM donors and centrifuged at 1,000 g for 15 minutes at 4° C. The supernatant was diluted 1:2 in 1% bovine serum albumin (BSA, Sigma, St. Louis, Mo.) in SFM and assayed by Bio-Plex Pro™ Human MMP 9-Plex Panel and Bio-Plex Pro™ Human Cytokine 27-plex Assay (Bio-Rad), using the MagPix® instrument (Luminex Corporation, Austin, Tex.).

Informed consent was obtained from patients recruited under a study approved by the Institutional Review Board at the University of Wisconsin-Madison and ascites was collected from patients with HGSOC (n=20) or benign conditions (n=4). Samples were diluted 1:2 in 1% BSA/PBS and assayed using a human MIP-1β DuoSet® ELISA (R&D Systems) following manufacturer's instructions.

PLSR model: The correlation between cytokine and MMP levels and HGSOC adhesion was analyzed by PLSR in SimcaP+v.12.0.1 (Umetrics; San Jose, Calif.) with mean-centered and variance-scaled data. The independent variable matrix (X) consisted of cytokine/MMP levels in culture, and the dependent variable matrix (Y) consisted of the percentage of tumor cells that adhered. R²Y, the coefficient of determination for Y, describes how well the model fits the behavior of Y. Q²Y measures the predictive value of the model based upon cross-validation. Components were defined sequentially, and if Q²Y increased significantly (>0.05) with the addition of the new component, that component was retained, and the algorithm continued until Q²Y no longer significantly increased.

Interventions in co-culture model: To determine if P-selectin played a role in adhesion, LP-9 in co-culture devices were treated with 10 μg/mL of anti-P-selectin blocking antibody or monoclonal mouse IgG1 isotype (BioLegend; San Diego, Calif.), or 10 μM of the small molecular inhibitor KF38789 (Tocris; Minneapolis, Minn.) or DMSO (0.0005% v/v) 1 hour prior to the addition of ovarian cancer cells. To examine the role of AAM-secreted cytokines on HGSOC adhesion, functional blocking antibodies against IL-13 (1 μg/mL), PDGF-BB (0.5 μg/mL), and MIP-1β (1 μg/mL) or 1 μg/mL polyclonal goat IgG isotype (R&D Systems) were added to adhesion models during the introduction of the AAM or control coverslip. To determine the impact of MIP-1β on mesothelial expression of SELP and HGSOC adhesion, mesothelial cells were treated with MIP-1β (Peprotech) for 24 hours. To investigate if MIP-1β regulated SELL′ through CCR5, LP-9 were treated with 100 ng/mL of MIP-1β and 20 μg/mL of CCR5 functional blocking antibody (R&D Systems) or monoclonal mouse IgG2b isotype (BioLegend) for 24 hours. To test the effectiveness of CCR5 therapeutics, LP-9 were treated with 100 ng/mL of MIP-1β and 10 μg/mL of maraviroc (Sigma) or DMSO (0.001% v/v) for 24 hours. To inhibit PI3K and MEK pathways, LP-9 were treated with 100 ng/mL of MIP-1β and 10 μM LY294002 or PD0325901 (Sigma), respectively, for 24 hours. To knockdown CD24, HGSOC cells were seeded overnight in a 6 well plate at 10,500 cells/cm² in complete growth media without penicillin/streptomycin, treated for 24 hours with 25 nM ON-TARGETplus™ CD24 or non-targeting pool siRNA (Dharmacon; Lafayette, Colo.), washed with PBS, and cultured in complete growth media containing penicillin/streptomycin for 72 hours prior to use in adhesion assay. To determine the impact of MIP-1β in ascites on HGSOC cell adhesion, LP-9 were treated with 10% (v/v) ascites treated with 1 μg/mL MIP-1β blocking antibody or 1 μg/mL polyclonal goat IgG isotype (R&D Systems) for 24 hours prior to addition of HGSOC.

HGSOC adhesion to P-selectin: Ibidi 2-well culture inserts (Ibidi; Munich, Germany) were coated with 50 μg/mL P-selectin Fc-chimera or rh IgG1-Fc (R&D) overnight at room temperature. HGSOC cells were stained with 5 μM CellTracker™ Green, dissociated using TrypLE, and seeded into each chamber of the insert at a concentration of 5,000 cells/40 μL. Cells were allowed to adhere for 3 hours, and percent adhesion was quantified as described above.

AKT and ERK phosphorylation: Following 0, 5, 15, 60, and 240 minutes of treatment with 100 ng/mL MIP-1β, LP-9 were lysed using the Bio-Plex® cell lysis buffer (Bio-Rad) according to manufacturer's instructions. Protein concentration was determined through a BCA assay. The levels of AKT (tAKT), pAKT(Thr308), pAKT(S473), ERK, and pERK1/2(Thr202/Tyr204, Thr185/Tyr187) were assayed using the Bio-Plex Pro Magnetic Cell Signaling Assay (Bio-Rad) and read using the MagPix® instrument. The measurement of each phosphorylation site was normalized to the total protein measurement for that sample.

Flow cytometry analysis: LP-9 were seeded at 93,500 cells/cm² and treated with 100 ng/mL MIP-1β for 24 hours. Cells were dissociated using trypsin (0.05%)-EDTA (0.02%) and stained with anti-P-selectin (20 μg/mL; R&D Systems) or mouse IgG1k isotype (Biolegend) and Alexa Fluor® 488 (1:1000).

HGSOC cells were dissociated using TrypLE and stained with CD24-FITC (1 μg/mL), CD162-Alexa Fluor® 647 (0.125 μg/mL), IgG1-FITC isotype, or IgG1-Alexa Fluor® 647 isotype (all BD Biosciences; San Jose, Calif.) in 2% BSA/PBS and 0.1% sodium azide. CD24 expression was analyzed on a BD Accuri™ C6 flow cytometer (BD; Franklin Lakes, N.J., USA), P-selectin expression was analyzed on a ThermoFisher Attune™ (UWCCC Flow Cytometry Laboratory), and CD162 expression was analyzed on a BD FACSCalibur™ flow cytometer (BD Biosciences, UWCCC Flow Cytometry Laboratory). Expression for each cell line was compared to isotype controls.

Immunofluorescent imaging: HGSOC were cultured at 10,500 cells/cm² overnight under normal growth conditions, fixed, and immunofluorescence was performed with anti-CD24 (BD Biosciences) or CD15s at a concentration of 1 μg/mL, Alexa Fluor® 488 goat anti-mouse secondary antibody, and imaged on the Zeiss Axio Observer.Z1. LP-9 were cultured at 62,500 cells/cm² overnight and treated with 100 ng/mL MIP-1β for 0, 5, 15, 60, and 240 minutes. Cells were fixed and immunofluorescence was performed with anti-NF-κβ p65 (Cell Signaling; 1:400 dilution), Alexa Fluor® goat anti-rabbit secondary antibody, and imaged as described above.

Paraffin-embedded samples of omental tissue from women over 18 years of age who underwent omentectomy or omental biopsy for HGSOC staging or non-HGSOC conditions were obtained from archived pathology samples through a protocol approved by the Institutional Review Board at the University of Wisconsin-Madison. Five micron sections were cut and deparaffinization and rehydration was performed prior to heat antigen retrieval using Universal Antigen Retrieval Solution (R&D Systems; Minneapolis, Minn.) according to manufacturer's instructions. Slides were blocked in tris buffered saline (TBS, Boston Bioproducts; Ashland, Mass.) supplemented with 1% BSA and 1% normal goat serum for 1 hour, then incubated overnight at 4° C. with antibodies (anti-calretinin (ab702, Abcam; Cambridge, United Kingdom) at 1:50, anti-P-selectin (15 μg/mL) diluted in the blocking solution. Goat anti-mouse Alexa Fluor® 488 and goat anti-rabbit Alexa Fluor® 594 (Life Technologies) were diluted in 1% BSA/TBS at a 1:300 dilution and incubated for 1 hour. Slides were sealed using ProLong® Diamond Antifade Mountant with DAPI. Imaging was performed as above.

Immunohistochemistry: Paraffin-embedded samples of mouse peritoneal wall, omentum, and mesentery were cut into 5 μM sections and deparaffinization and rehydration were performed prior to heat antigen retrieval using citrate buffer according to manufacturer's instructions. Endogenous peroxidase activity was blocked by incubating slides in 0.3% v/v hydrogen peroxide in methanol for 30 minutes. Slides were blocked overnight at 4° C. using diluted horse normal blocking serum in PBS from the VECTASTAIN® ABC-AP Universal Staining Kit (Vector Laboratories; Burlingame, Calif.). Sections were incubated with anti-CD62p (5 μg/mL; Biorbyt; Cambridge, United Kingdom) for one hour at room temperature. Diluted biotinylated universal secondary antibody solution (ABC-AP Universal Staining Kit) was prepared according to manufacturer's instructions and incubated on sections for 30 minutes. Sections were then stained with VECTASTAIN® ABC Reagent for 30 minutes and ImmPACT™ DAB Substrate (Vector Laboratories) for 5 minutes. Sections were then stained with Mayer's Hematoxylin Solution for 1 minute and mounted using ClearMount™ according to manufacturer's instructions. Imaging was performed as above.

HGSOC rolling: LP-9 were seeded to confluency at a concentration of 93,500 cells/cm² in a parallel-plate flow chamber (Ibidi μ-Slide VI 0.4, Ibidi). 24 hours later, LP-9 were washed with SFM and treated with 100 ng/mL MIP-1β or 0.1% BSA/PBS for an additional 24 hours, then washed with SFM. As a negative control, additional chambers were coated with 1% BSA/PBS. CaOV3 cells were stained with 5 μM CellTracker™ Green, dissociated using TrypLE, and suspended in SFM at 100,000 cells/mL. Spheroids of CellTracker™ Green stained CaOV3 were formed at a concentration of 50 cells/spheroid using the hanging drop method. Spheroids were then resuspended for the experiment at 700 spheroids/mL. A syringe pump (KD Scientific, Holliston, Mass.) was used to flow the cancer cells across the LP9 at a shear stress of 0.125 dyn/cm² for 30 seconds. Images were captured every 0.5 seconds using time lapse module of the Zen2 software. Instantaneous velocities of the cells were calculated using ImageJ software, and a cell was defined as rolling if it spent greater than five seconds at a mean velocity of less than 50% of the mean velocity of the cells on BSA. The rolling flux (cells/mm²/min) was calculated as the number of rolling cells divided by the area of the field of view and total capture time.

Analysis of CD24 in patient microarray data: The Kaplan-Meier plotter tool was used with the Gene Expression Omnibus and The Cancer Genome Atlas to calculate PFS and OS for stage II-IV and grades II and III patients with TP53 mutations, split into low and high CD24 based upon median expression.

Immunofluorescent imaging for P-selectin: LP-9 in co-culture devices were cultured with or without AAMs and treated with 1 μg/mL MIP-1β blocking antibody or polyclonal goat IgG isotype control (R&D Systems). LP-9 cells were fixed, and immunofluorescence was performed with anti-P-selectin (R&D Systems, 15 μg/mL) and Alexa Fluor® 488 goat anti-mouse secondary antibody. Nuclei were counterstained with Hoechst. Fixed cells were imaged at room temperature in PBS. Imaging was performed as described above, and P-selectin levels were quantified via mean fluorescence intensity using ImageJ.

Characterization of MIP-1β consumption in co-culture: To detect AAM secretion of MIP-1β, differentiated AAMs were cultured in SFM in co-culture devices for 24 hours. Conditioned media samples were diluted 1:4 in SFM and assayed using the MIP-1β DuoSet® ELISA as described in Methods.

Copy number analysis of CD24 in Cancer Cell Line Encyclopedia: Copy number estimates for CD24 in ovarian, endometrial, colorectal, and pancreatic cancer cell lines were obtained from the Cancer Cell Line Encyclopedia using the genome-wide human Affymetrix SNP Array 6.0

Statistical Analysis: All data are presented as mean±standard deviation. All experiments were performed at least twice, with unique AAM donors used for repeats of co-culture experiments. Statistical calculations (two-sided t-test, two-way ANOVA followed by Bonferroni corrected two-sided t-test, Kolmogorov-Smirnov test, log-rank test) were performed in GraphPad Prism software (La Jolla, Calif.).

### Example 1: AAMs Increase HGSOC Adhesion to Mesothelial Cells

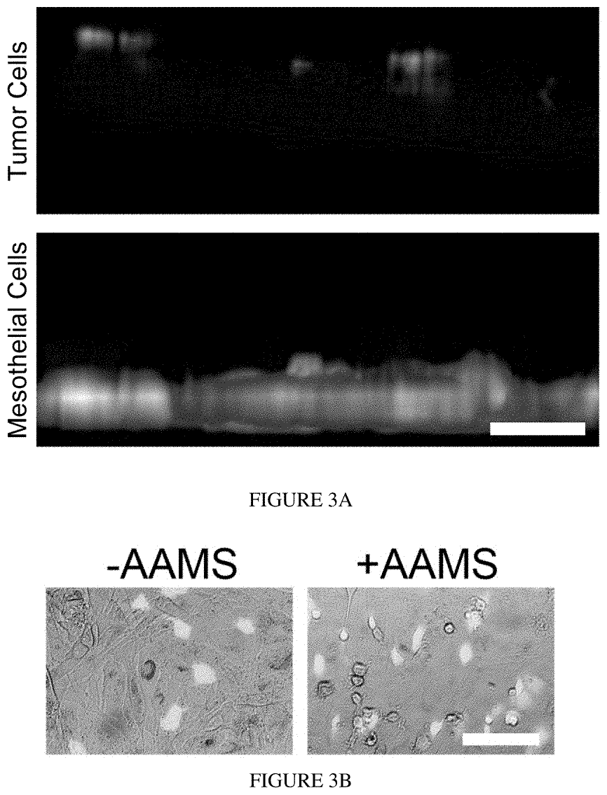

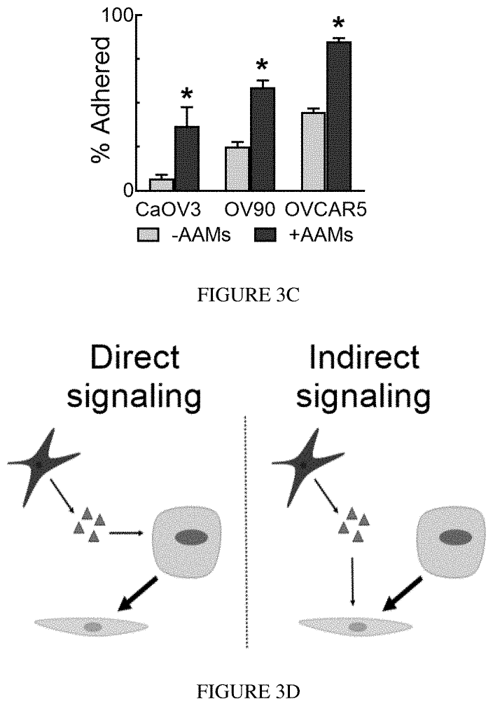

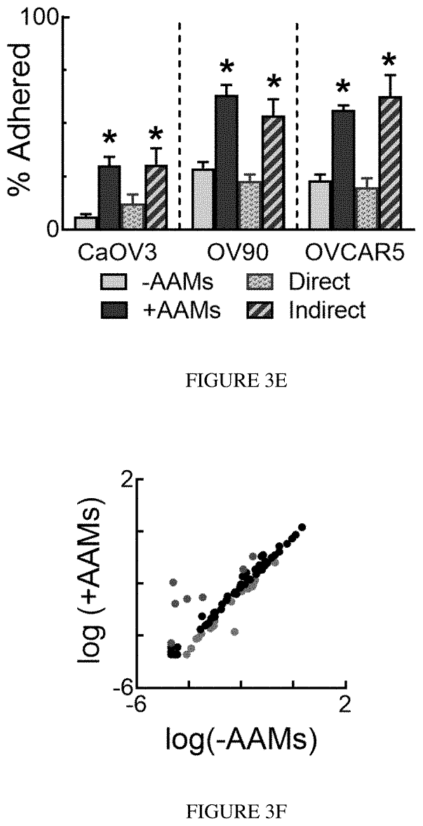

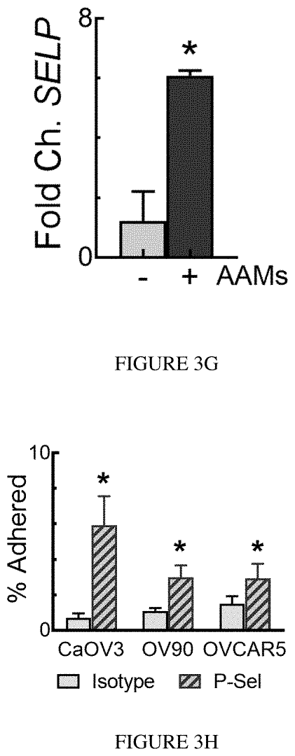

To examine the role of AAMs in HGSOC metastasis, an in vitro model of the peritoneal microenvironment was created that enables concentrated paracrine signaling (FIG. 2A). To simulate the microenvironment of a patient with metastatic disease, and hence an increase in AAM levels, LP-9 mesothelial cells were co-cultured with primary human AAMs for 24 hours (FIG. 2B). To mimic tumor cells floating in ascites, HGSOC cells in suspension were added to the device on top of the LP-9 and allowed to adhere for three hours (FIG. 2B). After removal of non-adherent cells, HGSOC that remained were adhered to the top of the mesothelial monolayer and had not yet invaded through the LP-9 (FIG. 3A). This is consistent with clinical observations that unlike other cancers, HGSOC does not infiltrate deeply. When AAMs were incorporated in the device, the percentage of HGSOC cells that adhered increased significantly (FIGS. 3A and 3B). Consistent with heterogeneity that is observed in HGSOC, baseline adhesion in the absence of AAMs varied among the three lines (FIG. 3C). However, all three showed increased adhesion with AAMs, suggesting that targeting the mechanism responsible for this increase could impact the extent of tumor metastasis in a broad group of patients. It was next examined whether AAM-secreted factors signaled to mesothelial cells (indirect) or tumor cells (direct) to increase HGSOC adhesion (FIG. 3D). To test for indirect signaling, LP-9 were co-cultured with AAMs for 24 hours, and then the AAMs were removed and fresh SFM media was added when HGSOC were seeded into the device. Direct signaling was tested by culturing LP-9 alone for 24 hours, and then adding AAMs and AAM-conditioned media when HGSOC were seeded. Results showed that HGSOC adhesion increased only in the indirect paradigm, suggesting that AAMs induced changes to mesothelial cells to enhance adhesion (FIG. 3E).

### Example 2: AAMs Regulate Mesothelial Expression of P-Selectin

Based on the observation that paracrine signals from AAMs to LP-9 enhanced adhesion, the inventors hypothesized that AAM-secreted factors upregulated extracellular matrix (ECM) or adhesion proteins on the mesothelial surface that HGSOC could then bind to. To test this hypothesis, mRNA was collected from LP-9 cultured alone or with AAMs and it was determined that 17 ECM/adhesion-related genes were downregulated, while seven genes were upregulated greater than two-fold (FIG. 3F and Table 1). Of particular interest was the increase in SELP (P-selectin), a member of the family of selectin cell adhesion molecules that other tumor cell types have been shown to bind but has been reported to be absent in mesothelial cells in vivo and in vitro. Validation by qRT-PCR across multiple AAM donors confirmed that LP-9 had a low expression of SELP, which was upregulated nearly six-fold during AAM co-culture (FIG. 3G). To test whether P-selectin contributed to HGSOC adhesion, examined adhesion of the HGSOC lines to adsorbed P-selectin was examined. All three HGSOC lines had significantly greater adhesion to adsorbed P-selectin compared to Fc control (FIG. 3H). To determine if HGSOC cells were adhering to the mesothelial cells through increased P-selectin, LP-9 was treated with a P-selectin blocking antibody prior to the addition of HGSOC cells. Consistent with the low basal expression of SELP, inhibition of P-selectin had no impact on basal adhesion (FIG. 3I). Blocking P-selectin countered the effect of AAM co-culture and returned levels of adhesion to those observed in cultures without macrophages (FIG. 3I). Together, this data suggest that AAMs upregulate SELP in mesothelial cells, which results in increased HGSOC adhesion.

TABLE 1
GENES THAT WERE DIFFERENTIALLY EXPRESSED
IN LP-9S CO-CULTURED WITH AAMS

| Gene | Fold Regulation |
|---|---|
| SELP | 2.50 |
| THBS1 | 2.93 |
| COL1A1 | 3.94 |
| MMP9 | 8.95 |
| SPP1 | 30.25 |
| ITGAM | 57.85 |
| ITGB2 | 457.24 |
| LAMA3 | −2.04 |
| CTNNB1 | −2.13 |
| HAS1 | −2.37 |
| ITGA1 | −2.49 |
| LAMA1 | −2.51 |
| MMP15 | −2.54 |
| COL7A1 | −2.61 |
| MMP12 | −2.78 |
| LAMB3 | −2.88 |
| MMP14 | −3.09 |
| ICAM1 | −3.09 |
| ITGA2 | −3.45 |
| MMP1 | −3.46 |
| MMP10 | −3.712 |
| COL16A1 | −3.75 |
| CLEC3B | −4.29 |
| ITGAL | −39.69 |

### Example 3: Partial Least Squares Regression (PLSR) Modeling Predicts a Role for AAM-Secreted MIP-1B in Enhanced HGSOC Adhesion

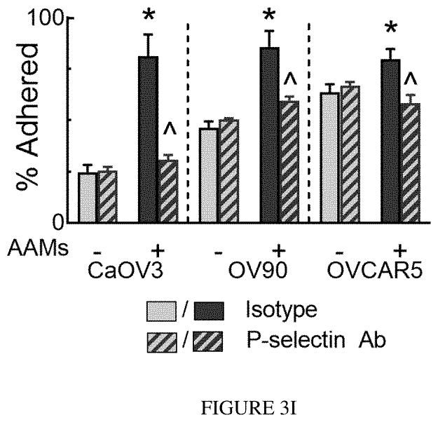

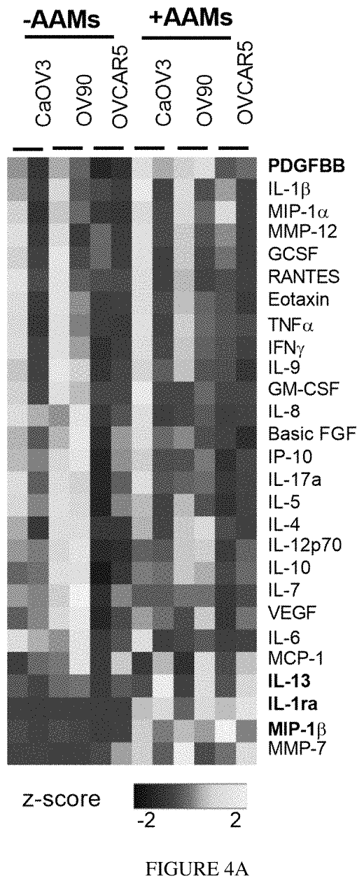

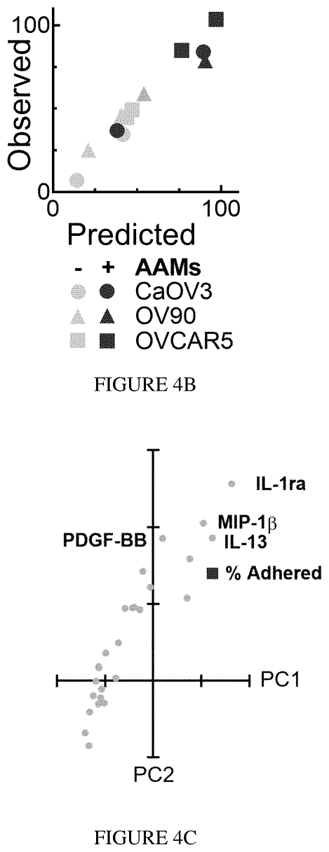

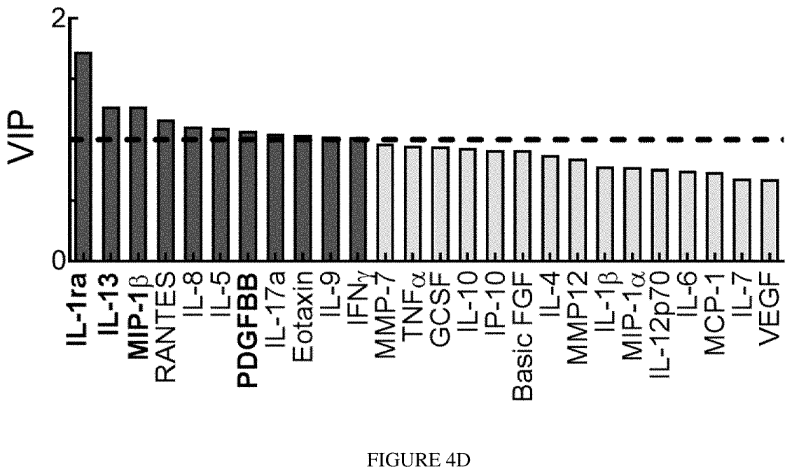

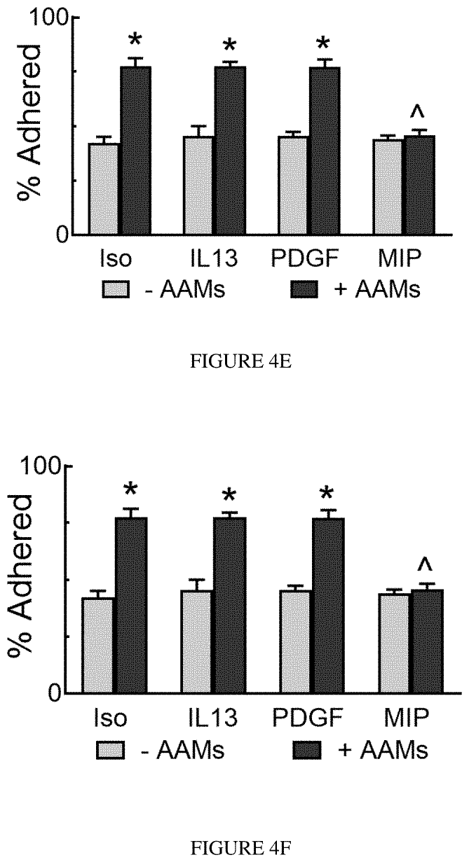

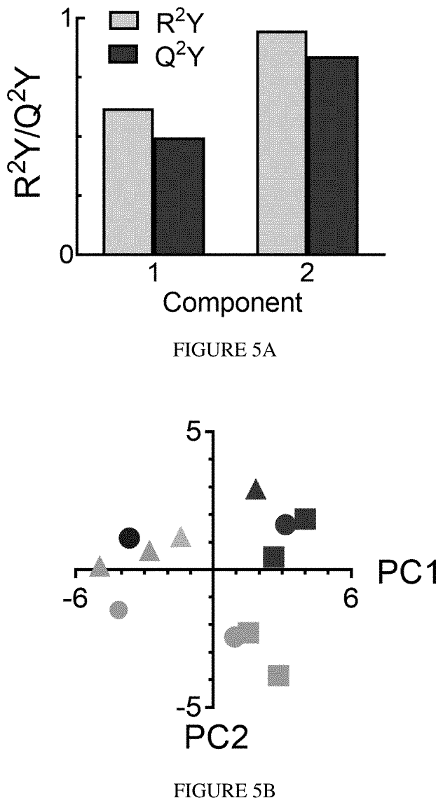

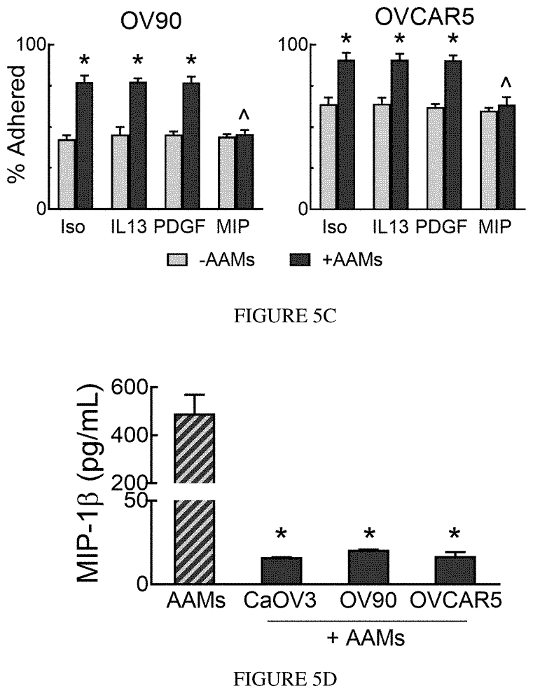

It was next determined which AAM-secreted molecule(s) were responsible for the increased adhesion of HGSOC. Media was collected from adhesion assays performed with two unique AAM donors and assayed for cytokines, chemokines, and matrix metalloproteinases (MMPs) (FIG. 4A and Table 2). Of the 36 screened ligands, 25 were detectable, with some ligands such as MIP-1β and MMP-7 elevated specifically when AAMs were present. Given the multivariate nature of the data, PLSR modeling was utilized to analyze the correlation between the concentration of secreted ligands and HGSOC adhesion. A two component PLSR model captured the co-variation between ligand secretion and adhesion (R²Y=0.95) and was highly predictive by cross-validation (Q²Y=0.84, FIG. 4B and FIG. 5A). Similar to our experimental observations above, conditions separated primarily based on difference across cell lines for the first component of the scores plot and based on the presence of AAMs in the second component (FIG. 5B). Analysis of the loadings (FIG. 4C) and variable importance in projection (VIP, FIG. 4D) identified four ligands (IL-13, MIP-1β/CCL-4, IL-1ra, and PDGF-BB) that clustered closely with adhesion while contributing significantly to the model (VIP>1).

TABLE 2
CYTOKINES, CHEMOKINES, AND MATRIX
METALLOPROTEINASES PRESENT IN ARRAY THAT
WERE NOT DETECTED IN ADHESION CULTURE
SAMPLES

| Cytokine and Chemokine Panel | Matrix Metalloproteinase Panel |
|---|---|
| IL-2 | MMP-1 |
| | MMP-2 |
| | MMP-3 |
| | MMP-8 |
| | MMP-9 |
| | MMP-10 |
| | MMP-13 |

To determine if these ligands were responsible for the increased adhesion, it was first examined what was known for each factor in HGSOC. IL-1ra was reported to decrease the extent of metastasis in mouse models of HGSOC, but the impact of IL-13, MIP-1β, and PDGF-BB are unknown, suggesting they may mediate the increased adhesion. To examine the impact of these AAM-secreted molecules, mesothelial cells were incubated with neutralizing antibodies against IL-13, PDGF-BB, or MIP-1β during co-culture with AAMs and addition of HGSOC cells. Inhibition of IL-13 and PDGF-BB had no impact on the enhancement of HGSOC adhesion observed with AAMs; however, inhibition of MIP-1β lowered HGSOC adhesion in the presence of AAMs to baseline levels, suggesting MIP-1β was necessary for the AAM-mediated effect on adhesion (FIG. 4E and FIG. 5C). Analysis of AAM-conditioned media confirmed that AAMs secrete MIP-1β, while HGSOC and mesothelial cells do not (FIG. 5D). Analysis of co-culture media suggested MIP-1β was consumed by the mesothelial and/or HGSOC cells, as levels were lower in co-culture samples compared to AAMs alone (FIG. 5D). To determine if MIP-1β was sufficient to increase adhesion in the absence of other AAM-secreted factors, LP-9 were treated with MIP-1β for 24 hours and assayed for adhesion. With MIP-1β treatment, all HGSOC lines had significantly increased adhesion, with levels comparable to the effects seen with AAM co-culture (FIG. 4F). Similar results were observed with an additional ascites-derived mesothelial cell line (LP-3, FIG. 5E).

### Example 4: MIP-1β Increases Mesothelial Cell Expression of SELP Through CCR5/PI3K

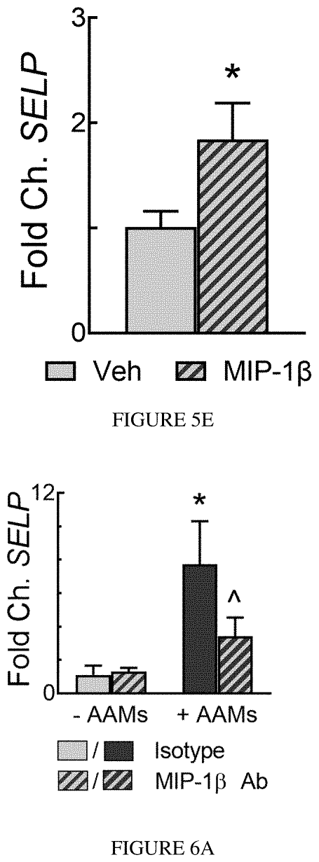

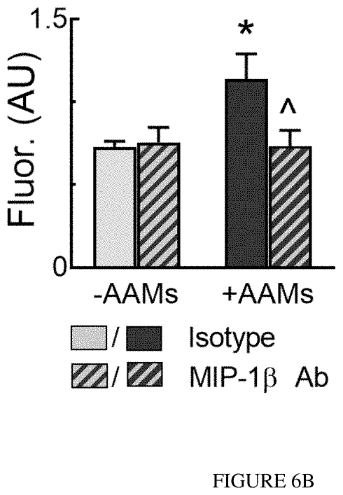

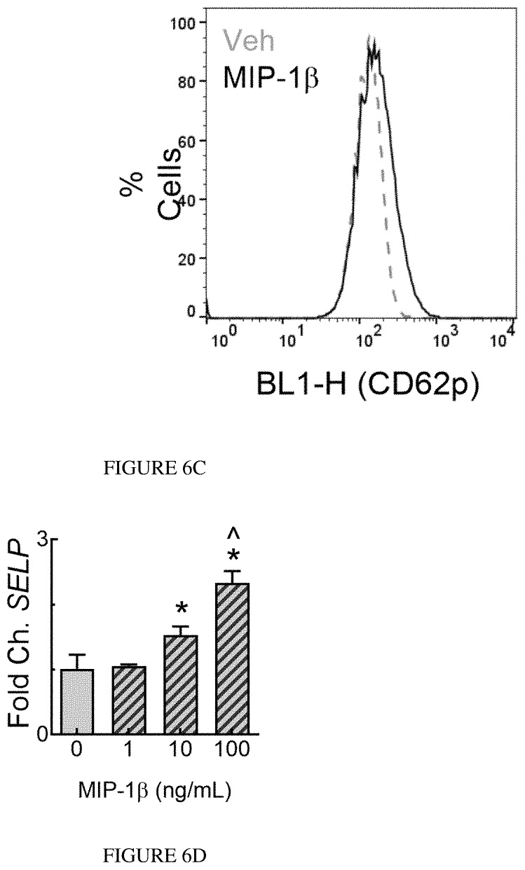

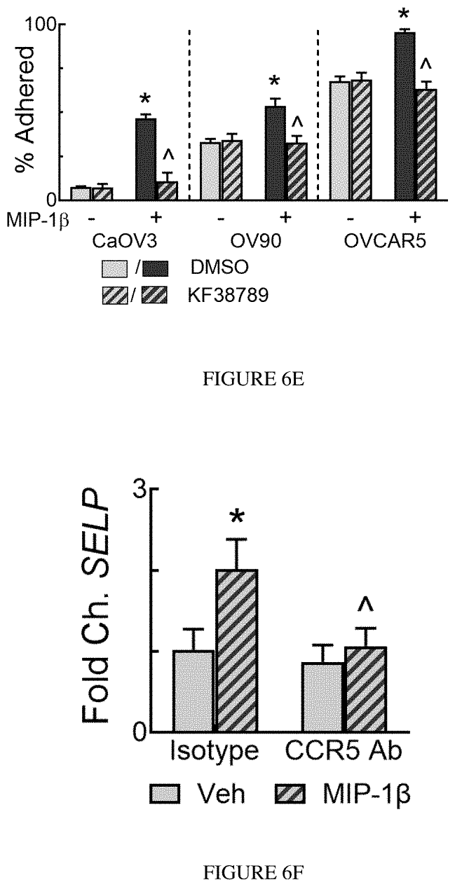

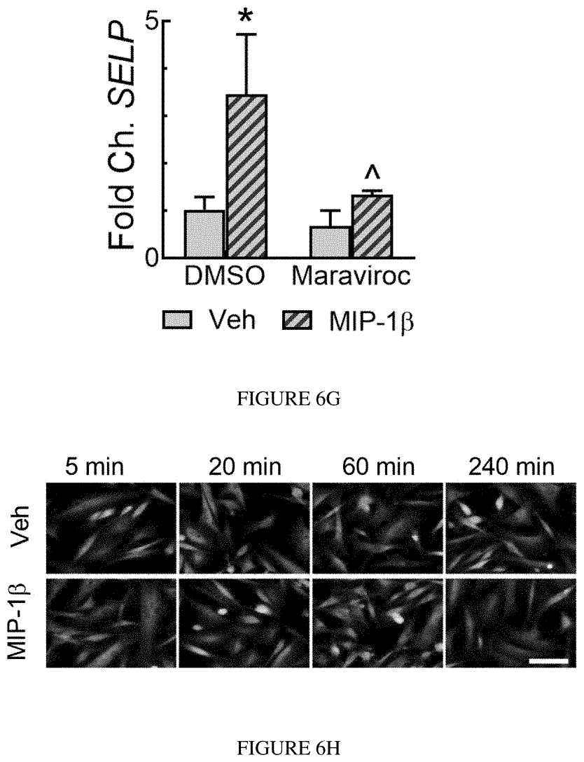

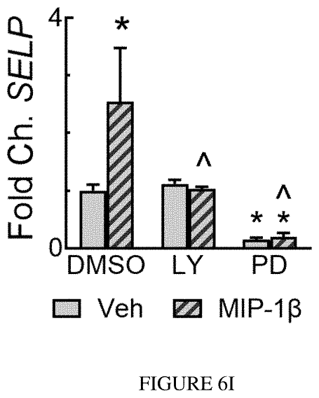

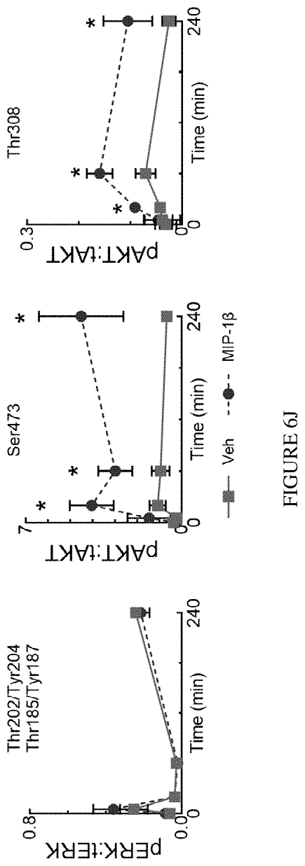

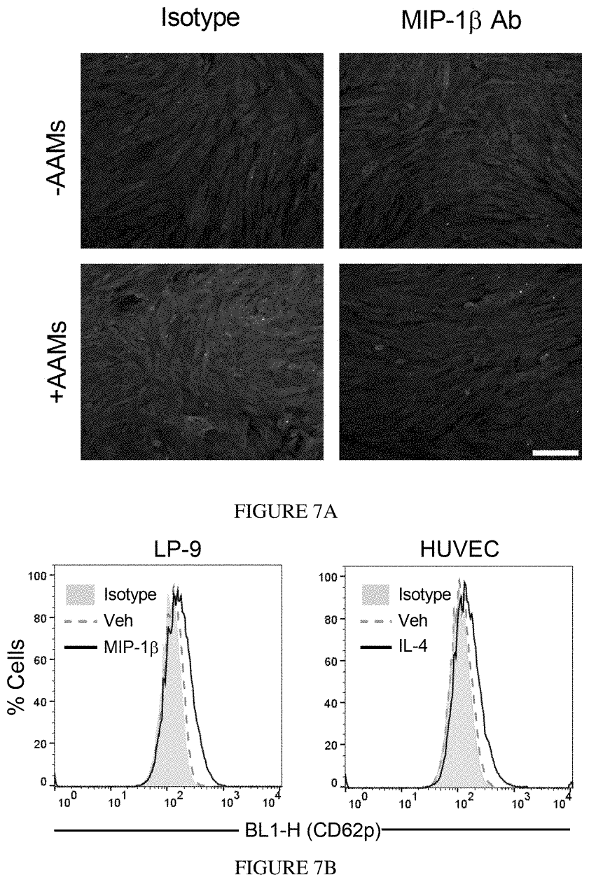

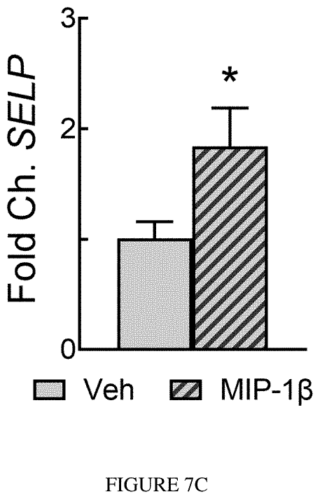

Given the observations that P-selectin and MIP-1β were each necessary for increased HGSOC adhesion in response to AAMs and that AAM-secreted factors upregulated SELP expression in mesothelial cells, it was hypothesized that AAM-secreted MIP-1β was responsible for increased SELF expression. To test this hypothesis, MIP-1β was inhibited in co-cultures of LP-9 and AAMs with a neutralizing antibody. P-selectin levels were examined in LP-9 at both the mRNA and protein level. qRT-PCR results showed that inhibition of MIP-1β significantly decreased SELP expression in LP-9 co-cultured with AAMs compared to isotype (FIG. 6A). Immunofluorescent imaging of P-selectin in LP-9 also showed that MIP-1β was necessary for upregulation of P-selectin by AAMs (FIG. 6B and FIG. 7A), and flow cytometry confirmed that MIP-1β increased surface expression of P-selectin on LP-9 (FIG. 6C). Treatment of LP-9 with increasing doses of MIP-1β resulted in a dose response of SELP expression, confirming that MIP-1β alone was sufficient to induce SELP (FIG. 6D, 7C). Similarly, treatment of LP-3 mesothelial cells with MIP-1β significantly increased SELP expression (FIG. 7B). Additionally, inhibition of P-selectin using the small molecular inhibitor KF38789 abrogated the increased adhesion that resulted from MIP-1β (FIG. 6E), further confirming that increased P-selectin increased HGSOC adhesion to mesothelial cells.

As a role for MIP-1β in the regulation of SELF expression has not been previously reported, the intracellular signaling pathways in mesothelial cells that may be responsible for this effect were investigated. Both CCR1 and CCR5 are receptors for MIP-1β; however, qRT-PCR analysis showed that LP-9 only expressed detectable levels of CCR5 (Table 3). To validate that MIP-1β signaled through CCR5 in LP-9, LP-9 was treated with 100 ng/mL MIP-1β and a CCR5 blocking antibody and determined that blocking CCR5 inhibited MIP-1β-stimulated expression of SELP (FIG. 6F). Clinically, CCR5 has been the target of drug development as it is an essential co-receptor for HIV entry. Maraviroc, a CCR5 allosteric modulator approved to treat HIV, was also effective in inhibiting MIP-1β-stimulated expression of SELF (FIG. 6G). CCR5 has been shown to activate NF-κβ, PI3K and MAPK, which can regulate SELF expression in other cell types. However, immunofluorescent staining of p65 showed no increase in nuclear co-localization upon treatment with MIP-1β (FIG. 6H), suggesting that NF-κβ does not play a role in P-selectin upregulation. Treatment with PD0325901, a MEK inhibitor, significantly decreased SELP expression in both vehicle and MIP-1β treated LP-9, suggesting that MEK activation is necessary for even the low basal expression of SELP in LP-9 (FIG. 6I). In contrast, inhibition of PI3K with LY294002 had no impact on basal SELP expression but significantly reduced the increase in SELP observed with MIP-1β treatment (FIG. 6I). Analysis of phosphorylation of ERK and AKT in response to MIP-1β treatment demonstrated no change in pERK, but an increase in pAKT at both Thr308 and Ser473 (FIG. 6J). Combined, these results suggest that MIP-1β activates CCR5 and PI3K to increase SELF transcription, and that therapy inhibiting CCR5 activation, such as maraviroc, may be effective in inhibiting SELP upregulation.

TABLE 3
EXPRESSION OF CCR1 AND CCR5
IN LP-9 MESOTHELIAL CELLS. ΔCT
DETERMINED RELATIVE TO GAPDH, N/D
INDICATES NOT DETECTABLE AFTER
50 ROUNDS OF AMPLIFICATION.

| | ΔCt |
|---|---|
| Receptor | (Avg ± SD) |
| CCR1 | N/D |
| CCR5 | 16.14 ± 0.17 |

### Example 5: HGSOC Cells Adhere to P-Selectin Through CD24

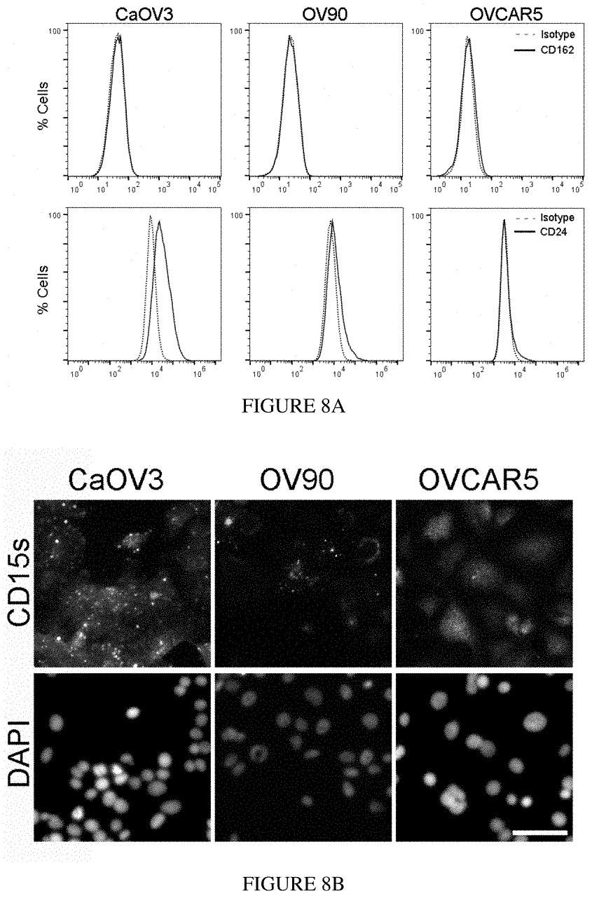

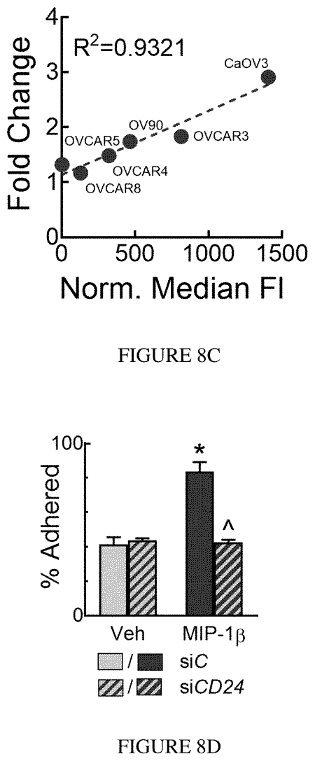

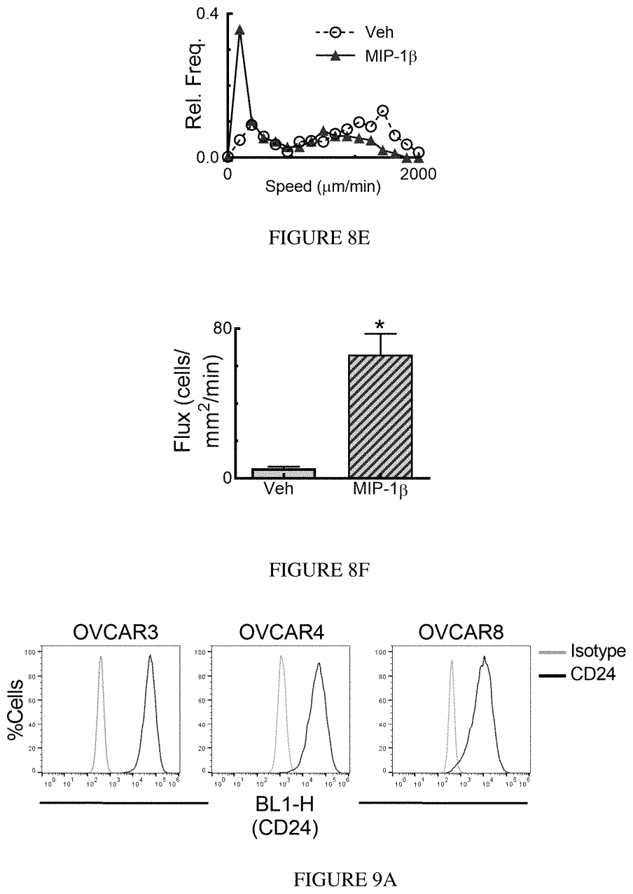

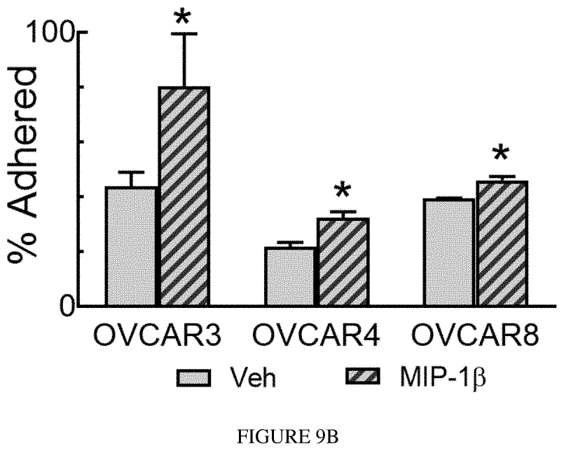

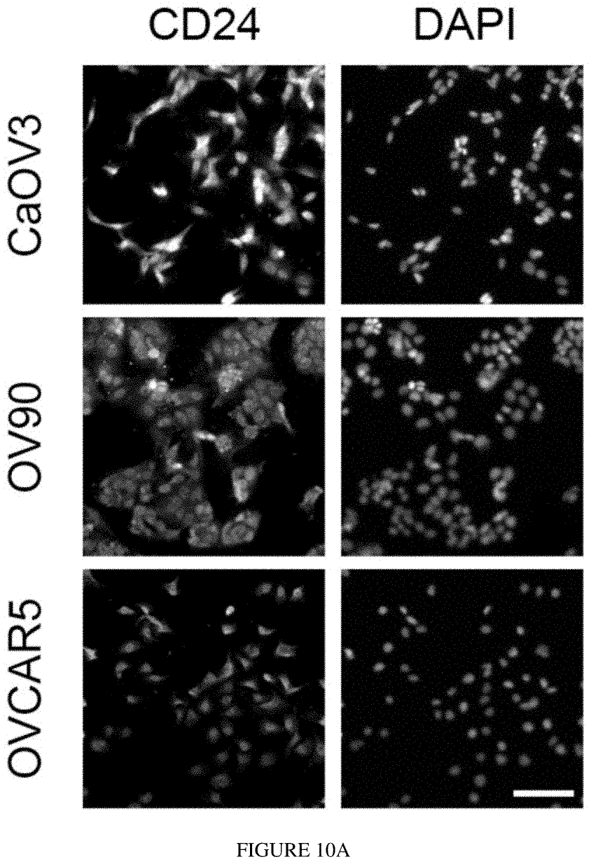

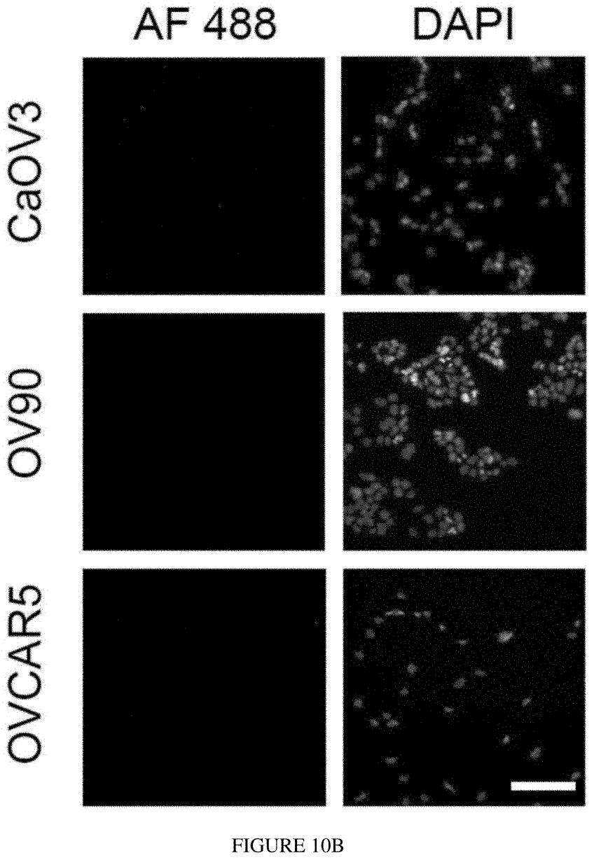

It was next determined which ligands are expressed on HGSOC cells to enable binding to P-selectin. The primary ligand for P-selectin, CD162 (PSGL-1), is expressed in neutrophils and lymphocytes, but has not been evaluated in the panel of HGSOC lines. Flow cytometry analysis indicated that none of the HGSOC lines in the panel expressed detectable levels of CD162 (FIG. 8, top panel). Alternatively, CD24 has been reported to act as a ligand for P-selectin and its overexpression is correlated with a poor prognosis in HGSOC patients. When the cell lines were examined, all expressed detectable levels of CD24, with the greatest surface expression in CaOV3 and the weakest in OVCAR5 (FIG. 8A bottom panel and FIG. 10). It has been shown that expression of siayl-Lewis(x) (CD15s) is necessary for CD24 to bind to P-selectin. When examined by immunofluorescent imaging, CaOV3 had the highest expression of CD15s and OVCAR5 had the lowest expression, similar to the pattern observed with CD24 (FIG. 8B). Given the variation in CD24/CD15s levels and the magnitude of increase in adhesion levels with AAM co-culture (FIG. 2C), the correlation between CD24 expression and the fold-change in percentage of cells that adhere with AAMs present was examined. We expanded our panel of HGSOC cells to six lines (FIGS. 9A and 9B) and identified a correlation between CD24 expression and the fold change in HGSOC adhesion to LP-9 treated with MIP-1β (FIG. 8C), suggesting that CD24 may be responsible for this effect. To examine this finding in more detail, we treated HGSOC cell lines with nontargeted (siC) or CD24-targeted (siCD24) siRNA and assayed adherence to LP-9 treated with MIP-1b or vehicle. Although CD24 knockdown had no impact on baseline adhesion, the loss of CD24 significantly reduced adhesion in the presence of AAMs for all HGSOC cell lines (FIG. 8D), suggesting a role for CD24 adhesion to P-selectin in the presence of AAMs and providing a potential explanation for the correlation between CD24 levels and prognosis.

### Example 6: Upregulation of P-Selectin on LP-9 Induces Rolling of HGSOC Cells Under Flow

While the analysis of the interactions between AAMs, mesothelial cells, and HGSOC presented in Examples 1-5 were conducted in static conditions, the peritoneal cavity is a complex environment subject to slow fluid flow as well as stagnant pockets. Selectins are best known for inducing rolling that slows leukocytes and supports integrin engagement. In particular, P-selectin has been shown to aid in the rolling of breast cancer cells along endothelial cells. To determine if this same rolling phenomenon occurred between HGSOC and mesothelial cells, the ability of MIP-1β-treated LP-9 to induce rolling of HGSOC cells was examined in a parallel plate flow chamber. In cell-free chambers coated with BSA or chambers with vehicle-treated LP-9, CaOV3 travelled at a similar free flow velocity (FIG. 8E) and rolling across the surface was not observed (FIG. 8F). However, CaOV3 exhibited slower velocities (FIG. 8E) and significantly more cell rolling on MIP-1β-treated LP-9 surfaces (FIG. 8F). The results of these dynamic flow experiments suggest that MIP-1β-upregulation of P-selectin in mesothelial cells increases rolling of HGSOC cells, which would translate to increased metastatic potential in regions of the peritoneal cavity that are subject to fluid flow.

### Example 7: MIP-1β Decreases Cell Speeds in Spheroids

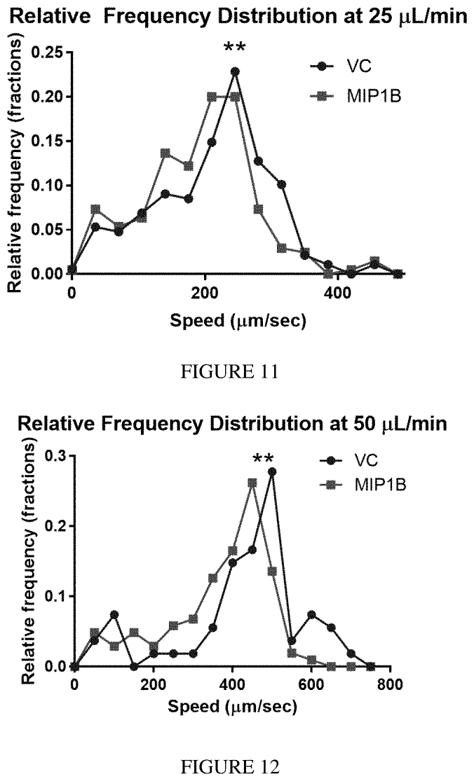

Since tumor cells in HGSOC spread as individual cells and as clumps of cells, the P-selectin method has been tested to see if it impacts rolling of aggregates. OV90s were stained with Cell Tracker™-green, and spheroids are formed in Aggrewells™. LP9s were seeded in collagen-coated ibidi microchannels at 93,500 cells/cm² and treated with 100 ng/mL MIP-1β; 24 hours after MIP-1β treatment, OV90 spheroids were flowed over LP9s at a constant flow rate/shear stress. The speed of the spheroid flow is calculated by tracking the distance traveled by the spheroids per frame in FIJI; statistical test is Kolmogorov-Smirnov test. FIG. 11 shows the results for 25 μL/min (0.0317 dyn/cm²), 700 spheroids/mL, 50 cells/spheroid, and FIG. 12 shows the results for 50 μL/min (0.0634 dyn/cm²), 700 spheroids/mL, 50 cells/spheroid. MIP-1β treatment of LP9s results in decreased cell speeds in spheroids at multiple shear stresses, consistent with P-selectin mechanism.

### Example 8: MIP-1β Increases P-Selectin Expression and Adhesion of HGSOC In Vivo

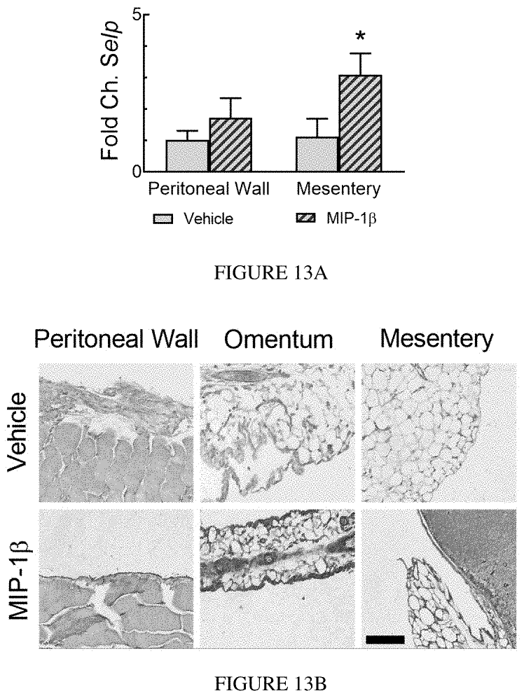

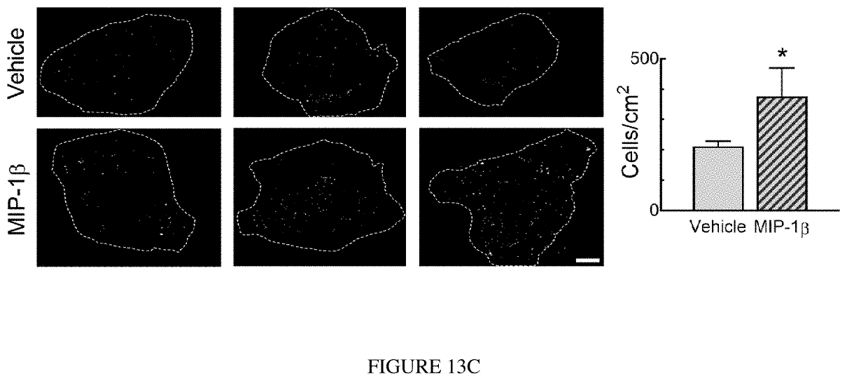

It was next investigated whether MIP-1β regulated P-selectin in vivo. C57/BL6 mice were injected with vehicle control or 1 μg MIP-1β. Analysis of Selp expression showed a small but not statistically significant increase in the peritoneal wall and a significant increase of nearly three-fold in the mesentery (FIG. 13A). However, this analysis measures the level of Selp throughout the entire tissue, but to increase adhesion P-selectin would need to be increased specifically in the mesothelial barrier. Therefore, immunohistochemistry for P-selectin was performed on the peritoneal wall, omentum, and mesentery. In all three tissues, P-selectin expression appeared elevated in the thin, flat layer of mesothelial cells lining the tissues (FIG. 13B). FIG. 15 is a control for FIG. 13B.

Next, it was determined whether this increase in P-selectin increased the adhesion of CD24+ HGSOC cells to peritoneal tissues. Adhesion of CaOV3 cells to excised peritoneal wall tissue was assayed for ex vivo and found to be significantly increased (FIG. 13C), suggesting that increased peritoneal tissue expression of P-selectin in response to MIP-1β also increases metastatic adhesion of HGSOC.

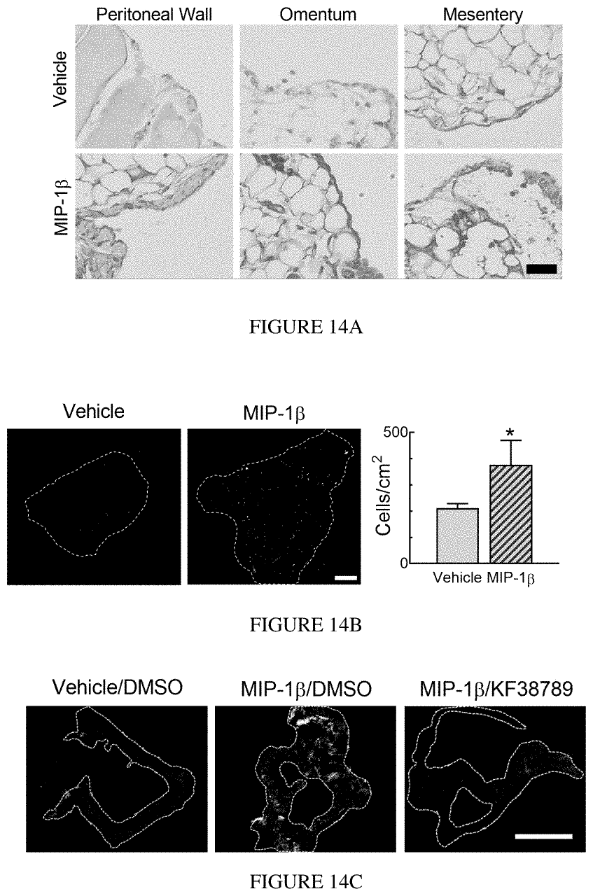

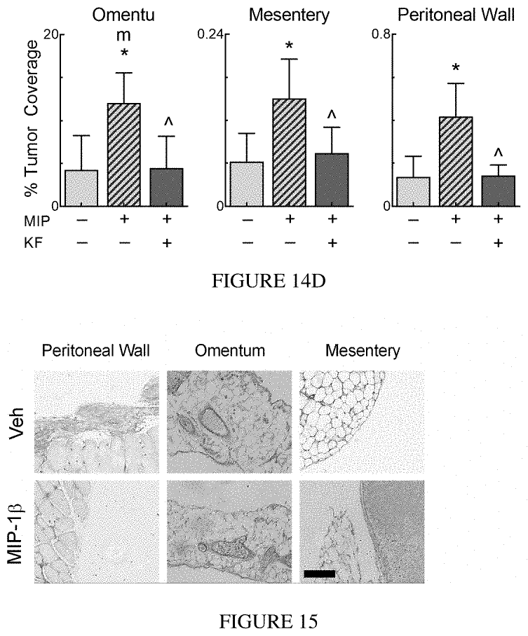

MIP-1β increases P-selectin in vivo and adhesion in vivo and ex vivo. FIG. 14A, IHC for P-selectin was performed on the peritoneal wall, omentum, and mesentery of mice that were intraperitoneally injected with vehicle or 1 μg MIP-1β. Scale bar, 100 μm. FIG. 14B, Ex vivo adhesion of CaOV3 to peritoneal wall biopsies from mice treated as in A. Scale bar, 1 mm. Images (left) and quantified adhesion (right) from n=3 mice. FIG. 14C and D, In vivo adhesion of ID8 to the peritoneal wall, omentum (shown in C), and mesentery was assayed after 90 minutes in mice intraperitoneally injected with vehicle control or 1 μg MIP-1β, followed by DMSO control or KF38789 (1 mg/kg, MIP-1β/KF38789). Scale bar, 0.5 cm. Data are Average+/−SD; *, P<0.05 vs. vehicle (B) or vehicle/DMSO (D); \hat{ }, P<0.05 vs. MIP-1β/DMSO by a two-sided t test (B) with Bonferroni correction (D).

### Example 9: MIP-1β, CD24, and P-Selectin are Upregulated During HGSOC Progression

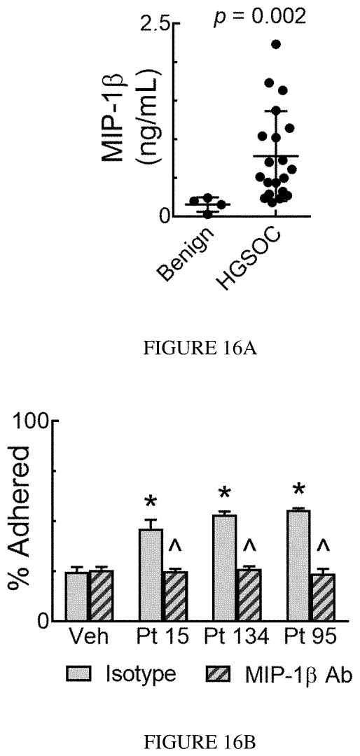

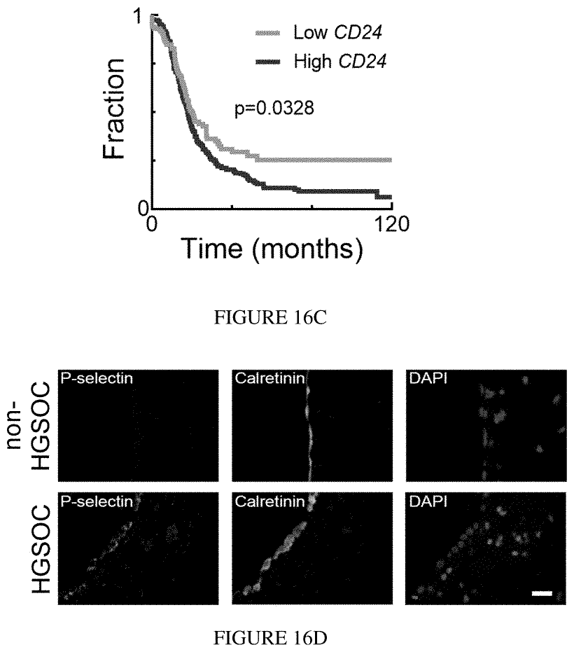

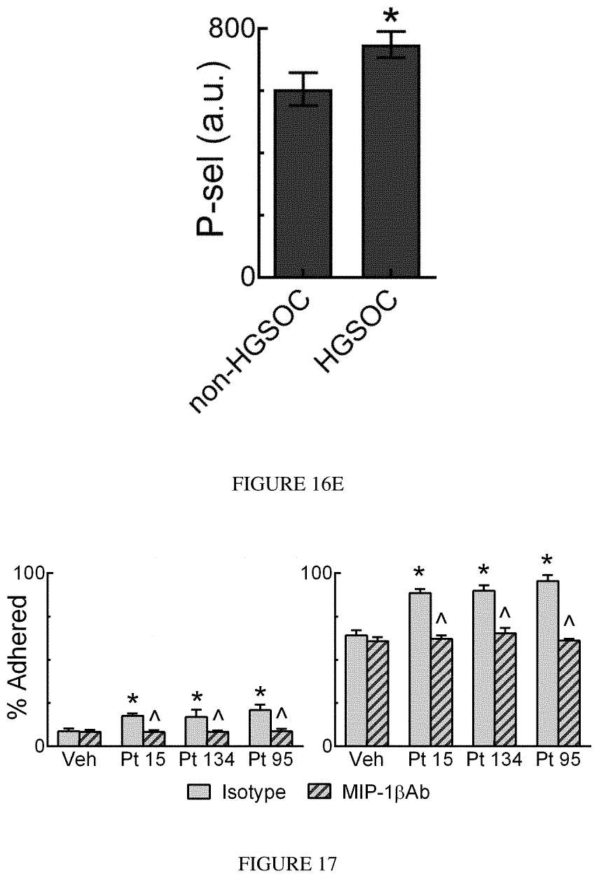

The mechanism was then examined in samples from HGSOC patients. Previous analyses of ascites in HGSOC showed that MIP-1β is elevated in the ascites of ovarian, fallopian tube, and peritoneal cancer patients compared to serum levels; however, to our knowledge, no studies have compared MIP-1β levels between ascites from HGSOC patients and those with benign conditions. Therefore, ascites were collected from patients undergoing surgery for benign conditions or HGSOC debulking and determined that MIP-1β was significantly elevated in HGSOC (FIG. 16A). Ascites is a complex mixture of multiple components, some of which could potentially inhibit the effects of MIP-1β on mesothelial cells. Therefore, LP-9 were treated with HGSOC ascites from the three patients with the highest levels of MIP-1β and tested the impact of ascites-derived MIP-1β on HGSOC adhesion. The results demonstrated that HGSOC adhesion increased significantly in response to ascites (FIG. 16B and FIG. 17), but adhesion decreased significantly when treated with a MIP-1β blocking antibody.

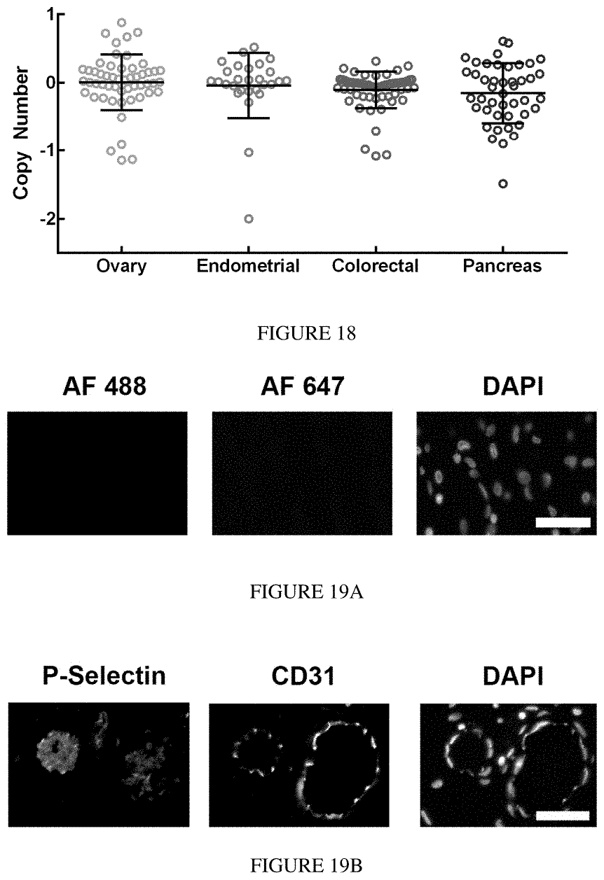

The expression of CD24 was examined across multiple cancer cell lines using the Cancer Cell Line Encyclopedia to compare CD24 expression in HGSOC and other cancers that metastasize to the peritoneum. Comparison of CD24 copy number in cell lines from ovarian, endometrial, colorectal and pancreatic cancers showed that, on average, these cancers had copies of the gene for CD24, however, ovarian cancer had the highest number (FIG. 18; Table 4).

TABLE 4
COPY NUMBER ESTIMATES FROM
OVARIAN CANCER CELL LINES

| Cell Line | Copy Number |
|---|---|
| OVCAR4 | 0.8824 |
| OVCAR8 | 0.6711 |
| KURAMOCHI | 0.587 |
| CaOV3 | 0.4018 |
| OV90 | −0.0161 |
| OVCAR3 | −0.1442 |
| OVCAR5 | −0.2681 |

Using the Kaplan Meier plotting tool and data from over 400 HGSOC patients in the Gene Expression Omnibus and The Cancer Genome Atlas, it was found that higher expression of CD24 was correlated with shorter progression free survival (PFS) in HGSOC patients (FIG. 16C and Table 5). This suggests that patients with tumor cells expressing CD24 have faster recurring diseases, possibly through enhanced metastatic spread due to P-selectin/CD24 interactions.

TABLE 5
PROGNOSTIC RESULTS FROM KAPLAN-MEIER
ANALYSIS OF CD24 EXPRESSION. STATISTICAL
COMPARISON BY LOG-RANK TEST.

| | low CD24 | high CD24 | P |
|---|---|---|---|
| Progression-free | 19.8 | 17.1 | 0.0328 |
| survival | n = 310 | n = 131 | |
| (months) | | | |

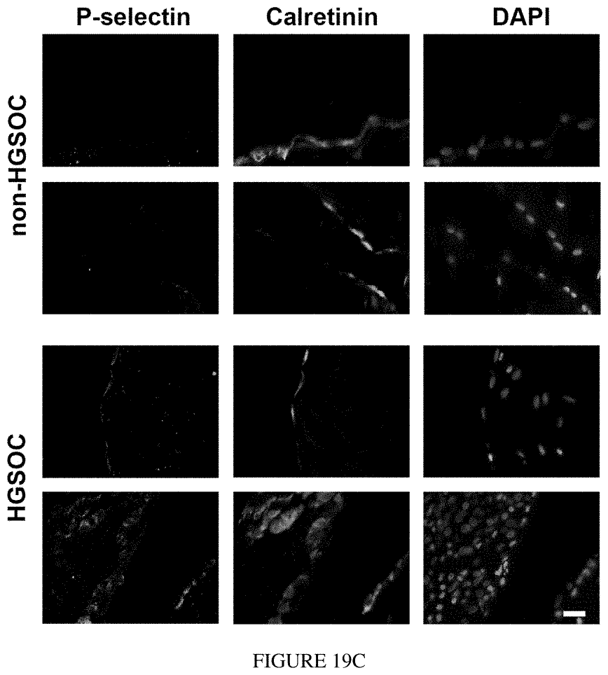

Finally, omental tissue was collected from non-HGSOC and HGSOC patients and stained for P-selectin and calretinin (a mesothelial marker). In omental samples that did not involve HGSOC, P-selectin was not detected in mesothelial cells (FIG. 16 and FIG. 19), consistent with prior reports. Some faint P-selectin positive regions were observed that were DAPI-negative; through staining with the endothelial cell marker CD31, this signal was confirmed to be from anuclear platelets in blood vessels (FIG. 18). In contrast, P-selectin was expressed in the omentum from HGSOC patients, and co-localized with the calretinin marker (FIG. 16D and FIG. 19). Quantification confirmed that mesothelial cells from HGSOC patients had significantly elevated P-selectin (FIG. 16E), suggesting that inhibiting P-selectin/CD24 interactions may be an effective method to slow or stop metastasis in HGSOC.

### Example 10: Xenograft Model Study

To confirm that xenograft models demonstrate increased MIP-1β and P-selectin, a longitudinal study of two different xenograft lines will be conducted. While there are differences between human tumors and mouse xenografts, macrophage infiltration has been observed in HGSOC xenografts and confirmed that some of these macrophages have an AAM phenotype. Additionally, it has been reported that mouse macrophages secrete MIP-1β. First, CaOV3 will be used, which consistently develops tumors but show a slow progression, with mice not meeting euthanasia requirements through at least 90 days. Second, OVCAR5 will be used, which develops tumors much more quickly (with mice requiring euthanasia by 26 days when untreated but was still sensitive to ouMIP-1β/P-selectin mechanism in vitro. Tumors will be initiated by i.p. injection of 5×10⁶ cells in 16 mice for each cell line. For CaOV3, half of the mice will be euthanized at 30 days and half at 60 days to assess tumor number, size and location, MIP-1β level in the peritoneal fluid by ELISA, and P-selectin in mesothelial cells by histology. Due to the more rapid progression with OVCAR5, half of the mice injected with OVCAR5 will be euthanized at 10 days and the other half at 20 days.

### Example 11: Xerograph in a P-Selectin Knockout Mouse

To confirm a role for P-selectin in tumor progression in the mouse xenograft, progression in xenografts in BALB/c scid mice will be compared to progression in a P-selectin knockout mouse (B6.129S7-Selp^{tm1Bay}/), backcrossed onto the BALB/c scid strain). Using the tumor cell line that induced the greatest change in P-selectin, tumor progression over time will be followed in the two animal models. Tumors will be initiated by i.p. injection of 5×10⁶ cells in 8 mice for each genotype. As i.p. tumors are difficult to assess through standard methods such as palpating and caliper measurements, either CaOV3 or OVCAR5 will be modified to stably express luciferase and examine tumor volume and location every 10 days by injecting luciferin i.p. and imaging tumor bioluminescence on an IVIS Spectrum. At the end of the experiment (90 days or when mice meet criteria for euthanasia), animals will be euthanized, assessed for total number of tumors, tumor size, and tumor location, and individual tumors will be examined by histology. From this experiment, it will be determined if the inability to upregulate P-selectin impacts long term progression.

### Example 12: Test the Impact of Therapies Against the MIP-1β/P-Selectin Mechanism on Tumor Progression

As a multi-cellular cascade, there are numerous opportunities to inhibit the impact of MIP-1β/P-selectin in order to slow or stop transcoelomic spread. For example, the in vitro studies demonstrated that a neutralizing antibody against MIP-1β, blocking antibody against CCR5, inhibition of PI3K, blocking antibody against P-selectin, or siRNA knockdown of CD24 were all effective in reducing macrophage-induced adhesion. However, in the more complex environment of the intact animal, these strategies may not be equivalent due to off-target effects limiting the dosing that can be used, effects of these inhibitors on other tumorigenic processes that may boost their efficacy, or practical limitations such as dosing frequency and cost. Therefore, a pre-clinical trial comparing the effects of two different approaches will be undertaken. Others have confirmed that peritoneal dissemination and ascites formation can be observed with HGSOC xenografts, making this an appropriate model for pre-clinical trials for HGSOC.

Both CaOV3 and OVCAR5 will be used to initiate xenografts in order to study the ability to alter tumor progression in both a slow and aggressive tumor system. CaOV3 and OVCAR5 will be modified to stably express luciferase, and i.p. tumors initiated as above. To mimic clinical presentation of HGSOC, treatment will begin after tumors have already established (8 animals per condition/cell line, 30 days for CaOV3^{luc+}, 10 days for OVCAR5^{luc+}). While it is more challenging to treat a tumor that is established in a mouse vs. immediately after initiation, this setup better mimics the clinical reality of HGSOC where patients are primarily diagnosed with advanced Stage III/IV disease. Animals will first be assessed by luciferin injection and bioluminescent imaging to confirm the presence of tumors and determine baseline size. Animals will be then treated with one of two inhibition strategies—inhibiting CCR5 to prevent the effects of MIP-1β or blocking P-selectin (detailed below, Table 6). Due to their different progression rates, bioluminescent imaging will be conducted every 5 days for OVCAR5^{luc+}, and every 10 days for CaOV3^{luc+}. After 90 days, or sooner if animals meet criteria outlined in Vertebrate Animal Care section, animals will be humanely euthanized and tumors excised. The number of tumors and location will be recorded, and tumor weight measured. Data will be analyzed to determine which inhibitors significantly delayed tumor progression, either in terms of tumor size/number (comparable to PFS) or time to euthanasia (comparable to OS).

TABLE 1
SUMMARY OF INHIBITION STRATEGIES
TO BE TESTED IN VIVO

| Inhibitor | Dose | Additional information |
|---|---|---|
| Maraviroc | 300 mg/L | Maraviroc, a CCR5 antagonist, has received FDA approval for |
| (Pfizer) | in drinking | HIV and is in trials for colon cancer (NCT01736813); side |
| | water (91) | effects that have been reported include liver problems and skin |
| | | reactions. Additional CCR5 antagonists are in clinical trials |
| | | for HIV (92) and diabetic nephropathy (PF-04634817, Phase |
| | | 2). |
| anti-mouse | 100 μg/ | Pre-clinical and clinical trials have been conducted for |
| P-selectin, | mouse; | inclacumab (a monoclonal antibody against P-selectin) for |
| RB40.34 | every 3 | saphenous vein graft failure following coronary artery bypass |
| (BD) | days (93) | surgery (94-96). While inclacumab had low efficacy for this |
| | | indication, it had a good safety profile for the 300 patients in |
| | | the trial (95), suggesting this therapy could potentially be |
| | | repurposed. Note that inclaclumab is specific to human P- |
| | | selectin; as P-selectin is expected on the host mesothelial cells, |
| | | we will use RB40.34. |

### Example 13: Impact of MIP-1β Induced P-Selectin on Colorectal Cancer Cell Adhesion

The experimental methods are reflective of those previously used to inhibit P-selectin, while used in an adhesion experiment with the colorectal cell lines LS411N and SW48.

The tissue culture plastic within the PDMS ring was coated with 1 μg of PureCol® Collagen I for 2 hours at room temperature. LP-9 were seeded into the PDMS rings to confluency (93,500 cells/cm² in 40 μL). Twenty-four hours after seeding, cells were washed with SFM, and 40 μL of fresh SFM containing either vehicle (0.1% BSA) or 100 ng/mL MIP-1β was added for 24 hours. To determine if P-selectin played a role in adhesion, LP-9 were treated with 10 μM of the small molecule P-selectin inhibitor KF38789 or DMSO (0.0005% v/v) 1 hour prior to the addition of colorectal cancer cells. The colorectal cancer cells (LS411N, SW48) were stained with 5 μM CellTracker™ Green CMFDA Dye, dissociated using TrypLE Select Enzyme, and seeded into devices at 10,000 cells/10 μL after 24 hours of mesothelial cell treatment with MIP-1β. Colorectal cancer cells were allowed to adhere for three hours, then coverslips were removed, and devices were washed twice with 2 mL PBS to remove non-adherent cells. Cells within the ring were fixed with 4% paraformaldehyde for 15 minutes, washed twice with PBS, and fluorescent colorectal cancer cells were imaged on a Zeiss Axio Observer.Z1 inverted microscope with an AxioCam 506 mono camera, Plan-NEOFLUOR 20× 0.4-NA air objective, and Zen2 software. Five images per well were captured, n=3 wells/condition. Percent adhesion was quantified by converting cells/image to total cells/area of the co-culture device and dividing by the number of HGSOC cells added to the device.

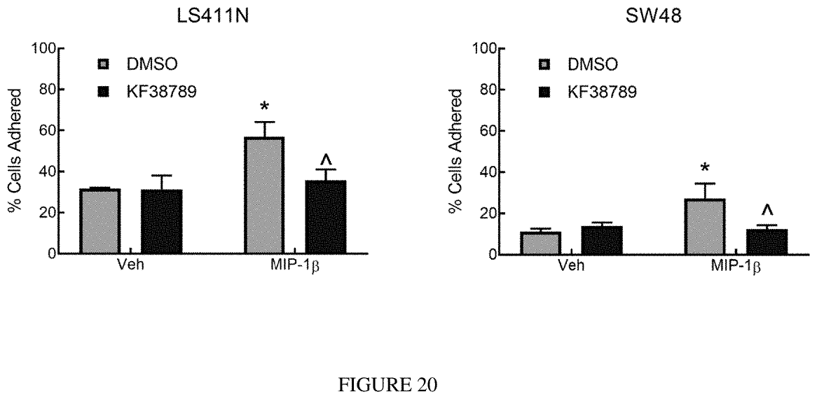

FIG. 20 shows that MIP-1β up-regulated P-selectin in LP-9 increased the adhesion of the colorectal cancer cell lines LS411N and SW48. Inhibition of P-selectin binding using KF38789 (10 μM) abrogates the increased adhesion from MIP-1β. These results reflect the same phenomena seen with the ovarian cancer cell lines.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of inhibiting metastasis in cancer, comprising
administering to a human subject diagnosed with a cancer of an organ of the peritoneal cavity a therapeutically effective amount of an inhibitor of P-selectin,
wherein the subject has a tumor positive for a ligand of P-selectin.

2. The method of claim 1, wherein the subject has a CD24+ or PSGL-1+ tumor.

3. The method of claim 1, wherein the organ of the peritoneal cavity is the ovaries, uterus, endometrium, cervix, small intestine, colon, anus, rectum, liver, gallbladder, pancreas, kidneys, or bladder.

4. The method of claim 3, wherein the subject is suffering from high-grade serous ovarian cancer.

5. The method of claim 4, wherein the high-grade serous ovarian cancer is stage III or stage IV cancer.

6. The method of claim 1, wherein the P-selectin inhibitor is a monoclonal antibody.

7. The method of claim 6, wherein the monoclonal antibody is crizanlizumab or inclacumab.

8. The method of claim 1, wherein the P-selectin inhibitor is rivipansel, or tinzaparin.

9. The method of claim 1, further comprising administering a chemotherapeutic agent.

10. The method of claim 9, wherein the chemotherapeutic agent is carboplatin, cisplatin, oxaliplatin, paclitaxel, docetaxel, olaparib, rucaparib, veliparib, or a combination thereof.

11. The method of claim 1, wherein the subject has had tumor removal surgery prior to administering.

12. The method of claim 1, wherein the subject has had neoadjuvant therapy prior to administering.

13. The method of claim 1, wherein the subject is in need of palliative care.

14. The method of claim 13, wherein inhibiting metastasis slows the incidence of bowel obstructions.

15. The method of claim 13, wherein the subject has chemotherapy-resistant cancer.
